SEQUENCE LISTING (2 sequences)

SEQ ID NO: 1 - protein, 225 aa, Homo sapiens
MAEKPKLHYFNARGRMESTRWLLAAAGVEFEEKFIKSAEDLDKLRNDGYLMFQQVPMVEIDGMKLVQTRAILNYIASKYNLYGKDIKERALIDMYIEGIADLGEMILLLPVCPPEEKDAKLALIKEKIKNRYFPAFEKVLKSHGQDYLVGNKLSRADIHLVELLYYVEELDSSLISSFPLLKVTHFTAQRGSPTSPILGSCIWALRLTKFCPSLLQAFSAPRSPK

SEQ ID NO: 2 - DNA, 1276 bp, Homo sapiens
attgagaggaacaaagagcttataaatacattaggacctggaattcagttgtcgagccaggacggtgacagcgtttaacaaagcttagagaaacctccaggagactgctatcatggcagagaagcccaagctccactacttcaatgcacggggcagaatggagtccacccggtggctcctggctgcagctggagtagagtttgaagagaaatttataaaatctgcagaagatttggacaagttaagaaatgatggatatttgatgttccagcaagtgccaatggttgagattgatgggatgaagctggtgcagaccagagccattctcaactacattgccagcaaatacaacctctatgggaaagacataaaggagagagccctgattgatatgtatatagaaggtatagcagatttgggtgaaatgatcctccttctgcccgtatgtccacctgaggaaaaagatgccaagcttgccttgatcaaagagaaaataaaaaatcgctacttccctgcctttgaaaaagtcttaaagagccatggacaagactaccttgttggcaacaagctgagccgggctgacattcatctggtggaacttctctactacgtcgaggagcttgactccagtcttatctccagcttccctctgctgaaggccctgaaaaccagaatcagcaacctgcccacagtgaagaagtttctacagcctggcagcccaaggaagcctcccatggatgagaaatctttagaagaagcaaggaagattttcaggttttaataacgcagtcatggaggccaagaacttgcaataccaatgttctaaagttttgcaacaataaagtactttacctaagtgttgattgtgcctgttgtgaagctaatgaactctttcaaattatatgctaattaaataatacaactcctattcgctgacttagttaaaattgatttgttttcattaggatctgatgtgaattcagatttccaatcttctcctagccaaccattttcctggaattaaaaattcagtaaaaaaggaaactatagattatgtggtttgtttgacttttccaagaattgtcccgtaacatacaatttgtcatacaatctattaaaatgtcaatgtagaaatgcacttctgacattttcaggtatgcacaggagaagagttaccatcctggataatggcataaagacattttcttcttttcctggacagtcattttatttctgataaaagcgttctttcttatgcaaaaaaaaaaaaaaaaaa

(19) United States
(12) Patent Application Publication
(10) Pub. No.: US 2017/0321283 A1
(43) Pub. Date: Nov. 9, 2017

(54) METHODS OF DIAGNOSING CANCER AND OF PREDICTING RESPONSE OF CANCER TO DENDROGENIN A TREATMENT

(71) Applicants: INSERM (INSTITUT NATIONAL DE LA SANTÉ ET DE LA RECHERCHE MÉDICALE), Paris (FR); UNIVERSITÉ PAUL SABATIER TOULOUSE III, Toulouse (FR); AFFICHEM, Toulouse (FR)

(72) Inventors: Marc POIROT, Toulouse (FR); Sandrine SILVENTE-POIROT, Toulouse (FR); Emmanuel NOGUER, Toulouse (FR)

(73) Assignees: INSERM (Institut National De La Sante Et De La Recherche Medicale), Paris (FR); Universite Paul Sabtier Toulouse III, Toulouse (FR); Affichem, Toulouse (FR)

(21) Appl. No.: 15/526,778

(22) PCT Filed: Nov. 16, 2015
(86) PCT No.: PCT/EP2015/076697

§ 371 Date: May 15, 2017

(30) Foreign Application Priority Data
Nov. 17, 2014 (EP) 14306817.9

(51) Int. Cl. C12Q 1/68; G01N 33/574

(52) U.S. Cl. CPC C12Q 1/6886; G01N 33/57415; G01N 33/57484; C12Q 2600/118; C12Q 2600/106; C12Q 2600/158

(57) ABSTRACT

The present invention relates to methods for the diagnosis and the treatment of cancer, in particular breast cancer. In particular, the present invention relates to a method of diagnosing cancer in a subject comprising the steps of i) determining the expression level of hGSTA1 in a tumor sample obtained from the subject, ii) comparing the expression level determined at step i) with its predetermined reference value and ii) concluding that the subject suffers from a cancer when the expression level of hGSTA1 is lower than its predetermined reference value.

### FIELD OF THE INVENTION

The present invention relates to methods for the diagnosis and the treatment of cancer, in particular breast cancer.

### BACKGROUND OF THE INVENTION

Several recent studies have shown that cholesterol metabolism was dynamically associated with cancers. Indeed, cholesterol metabolites with tumor promoter (TP) properties such as 27-hydroxycholesterol^{1,2}, cholesteryl fatty acid esters³⁻⁵ and a metabolite of cholestane-3β,5α,6β-triol^{6,7} have been described, supporting the role of cholesterol in cancer development. Importantly, the argument at the molecular level in favor of a unique positive implication of cholesterol in tumor promotion was counterbalanced by the discovery of dendrogenin A (DDA), which is a cholesterol metabolite with tumor suppressor (TS) properties⁸⁻¹⁰. On the one hand, overloading a cell with cholesterol or in its TP metabolites may favor tumor development and would suggest that the use of anti-cholesterol drugs may protect against cancer. On the other hand, the existence of a TS would suggest the introduction of a risk factor for cancer development if cholesterol neosynthesis is blocked by anti-cholesterol drugs.

DDA is a new endogenous modulator of LXR that controls tumor cell re-differentiation at nM doses^{10,11} and induces autophagic cell death in melanoma^{8,9} and in acute myeloid leukemia cells at μM concentrations in vitro and in vivo. DDA induces differentiation of multipotent normal progenitor cells of neuronal origins at nM concentrations suggesting an active role for this molecule in cell fate¹². The identification of cholesterol metabolites with opposite properties suggests a possible ratio between these TP and TS levels, and modification of this ratio in favor of a TP may promote cancer or alternatively an in increase in the ratio in favor of a TS may protect against cancer. It is thus important to identify the mechanism by which DDA is synthesized. DDA (5α-hydroxy-6β-[2-(1H-imidazol-4-yl)ethylamin]-cholestan-3β-ol) is the first steroidal alkaloid discovered to date in mammals. DDA resulted from the conjugation of 5,6α-epoxy-cholesterol (5,6α-EC) with histamine (HA). It was shown that the chemical and biochemical synthesis required a catalyst or an enzyme. Indeed, as opposed to most epoxide bearing chemicals, 5,6α-EC did not react spontaneously with nucleophilic functional groups such as amines^{11,13}. It was shown that mammalian tissue homogenates produced DDA from 5,6α-EC and HA, but this transformation was lost when homogenates were denatured by heat or proteases: evidence for the requirement of a proteinaceous enzyme for DDA biosynthesis. Thus, identification of the DDA synthase (DDAS) is important because: 1) it catalyzes a new type of enzymatic reaction of hisaminylation of 5,6α-EC; 2) it will help to understand if the deficiencies in DDA content in breast cancer are due to a lack in DDAS or not; 3) it will enable the identification of DDA producing cells in the breast.

### SUMMARY OF THE INVENTION

The present invention relates to methods for the diagnosis and the treatment of cancer, in particular breast cancer. In particular, the present is defined by the claims.

### DETAILED DESCRIPTION OF THE INVENTION

Dendrogenin A (DDA) is a new mammalian endogenous cholesterol metabolite with tumor suppressor properties. DDA is produced by a yet unknown enzyme catalyzing the histaminylation of 5,6α-epoxy-cholesterol (5,6α-EC). The inventors report here that DDA synthase (DDAS) is the human glutathione S-transferase A1 (hGSTA1). The catalytic efficiency of hGSTA1 was higher for 5,6α-EC and histaminylation than for its xenobiotic substrate glutathione suggesting that its main function was DDAS activity. In the breast, DDAS is selectively expressed in the cytoplasm of epithelial cells from lactiferous ducts and lobules. DDAS was exclusively found in cells at the origin of luminal breast cancers. In contrast, in breast tumors and cancer cell lines, the expression of DDAS and the production of DDA were drastically reduced. This study established the identification of DDAS as hGSTA1 and that its lack of expression was associated with oncogenic processes leading to luminal breast cancers.

### Diagnostic Methods of the Invention

Accordingly, an object of the present invention relates to a method of diagnosing cancer in a subject comprising the steps of i) determining the expression level of hGSTA1 in a tumor sample obtained from the subject, ii) comparing the expression level determined at step i) with its predetermined reference value and ii) concluding that the subject suffers from a cancer when the expression level of hGSTA1 is lower than its predetermined reference value.

Typically, the cancer may be selected from the group consisting of bile duct cancer (e.g. periphilar cancer, distal bile duct cancer, intrahepatic bile duct cancer), bladder cancer, bone cancer (e.g. osteoblastoma, osteochrondroma, hemangioma, chondromyxoid fibroma, osteosarcoma, chondrosarcoma, fibrosarcoma, malignant fibrous histiocytoma, giant cell tumor of the bone, chordoma, lymphoma, multiple myeloma), brain and central nervous system cancer (e.g. meningioma, astocytoma, oligodendrogliomas, ependymoma, gliomas, medulloblastoma, ganglioglioma, Schwannoma, germinoma, craniopharyngioma), breast cancer (e.g. ductal carcinoma in situ, infiltrating ductal carcinoma, infiltrating, lobular carcinoma, lobular carcinoma in, situ, gynecomastia), Castleman disease (e.g. giant lymph node hyperplasia, angiofollicular lymph node hyperplasia), cervical cancer, colorectal cancer, endometrial cancer (e.g. endometrial adenocarcinoma, adenocanthoma, papillary serous adnocarcinroma, clear cell), esophagus cancer, gallbladder cancer (mucinous adenocarcinoma, small cell carcinoma), gastrointestinal carcinoid tumors (e.g. choriocarcinoma, chorioadenoma destruens), Hodgkin's disease, non-Hodgkin's lymphoma, Kaposi's sarcoma, kidney cancer (e.g. renal cell cancer), laryngeal and hypopharyngeal cancer, liver cancer (e.g. hemangioma, hepatic adenoma, focal nodular hyperplasia, hepatocellular carcinoma), lung cancer (e.g. small cell lung cancer, non-small cell lung cancer), mesothelioma, plasmacytoma, nasal cavity and paranasal sinus cancer (e.g. esthesioneuroblastoma, midline granuloma), nasopharyngeal cancer, neuroblastoma, oral cavity and oropharyngeal cancer, ovarian cancer, pancreatic cancer, penile cancer, pituitary cancer, prostate cancer, retinoblastoma, rhabdomyosarcoma (e.g. embryonal rhabdomyosarcoma, alveolar rhabdomyosarcoma, pleomorphic rhabdomyo sarcoma), salivary gland cancer, skin cancer (e.g. melanoma, nonmelanoma skin cancer), stomach cancer, testicular cancer (e.g. seminoma, nonseminoma germ cell cancer), thymus cancer, thyroid cancer (e.g. follicular carcinoma, anaplastic carcinoma, poorly differentiated carcinoma, medullary thyroid carcinoma, thyroid lymphoma), vaginal cancer, vulvar cancer, and uterine cancer (e.g. uterine leiomyosarcoma). In some embodiments, the cancer is breast cancer.

The term “tumor sample” means any tissue tumor sample derived from the subject. Said tissue sample is obtained for the purpose of the in vitro evaluation. The sample can be fresh, frozen, fixed (e.g., formalin fixed), or embedded (e.g., paraffin embedded). In some embodiments the tumor sample may result from the tumor resected from the subject. In some embodiments, the tumor sample may result from a biopsy performed in the primary tumour of the subject or performed in metastatic sample distant from the primary tumor of the subject. For example an endoscopical biopsy performed in the bowel of the subject affected by a colorectal cancer.

As used herein, the terms “hGSTA1” has its general meaning in the art and refer to the human glutathione S-transferase alpha 1. Exemplary amino acid sequences for hGSTA1 is SEQ ID NO:1. Exemplary nucleic acid sequences for hGSTA1 is SEQ ID NO: 2. The term also include hGSTA1 variants include proteins substantially homologous to native hGSTA1, i.e., proteins having one or more naturally or non-naturally occurring amino acid deletions, insertions or substitutions (e.g., hGSTA1 derivatives, homologs and fragments). The amino acid sequence of a hGSTA1 variant can be at least about 80% identical to a native hGSTA1, or at least about 90% identical, or at least about 95% identical with SEQ ID NO:1.

| SEQ ID NO: 1: hGSTA1_homo sapiens_amino acid sequence | |
|---|---|
| 1 | maekpklhyf nargrmestr wllaaagvef eekfiksaed ldklrndgyl mfqqvpmvei |
| 61 | dgmklvqtra ilnyiaskyn lygkdikera lidmyiegia dlgemilllp vcppeekdak |
| 121 | lalikekikn ryfpafekvl kshgqdylvg nklsradihl vellyyveel dsslissfpl |
| 181 | lkvthftaqr gsptspilgs ciwalrltkf cpsllqafsa prspk |
| SEQ ID NO: 2: hGSTA1_homo sapiens_nucleic acid sequence | |
| 1 | attgagagga acaaagagct tataaataca ttaggacctg gaattcagtt gtcgagccag |
| 61 | gacggtgaca gcgtttaaca aagcttagag aaacctccag gagactgcta tcatggcaga |
| 121 | gaagcccaag ctccactact tcaatgcacg gggcagaatg gagtccaccc ggtggctcct |
| 181 | ggctgcagct ggagtagagt ttgaagagaa atttataaaa tctgcagaag atttggacaa |
| 241 | gttaagaaat gatggatatt tgatgttcca gcaagtgcca atggttgaga ttgatgggat |
| 301 | gaagctggtg cagaccagag ccattctcaa ctacattgcc agcaaataca acctctatgg |
| 361 | gaaagacata aaggagagag ccctgattga tatgtatata gaaggtatag cagatttggg |
| 421 | tgaaatgatc ctccttctgc ccgtatgtcc acctgaggaa aaagatgcca agcttgcctt |
| 481 | gatcaaagag aaaataaaaa atcgctactt ccctgccttt gaaaaagtct taaagagcca |
| 541 | tggacaagac taccttgttg gcaacaagct gagccgggct gacattcatc tggtggaact |
| 601 | tctctactac gtcgaggagc ttgactccag tcttatctcc agcttccctc tgctgaaggc |
| 661 | cctgaaaacc agaatcagca acctgcccac agtgaagaag tttctacagc ctggcagccc |
| 721 | aaggaagcct cccatggatg agaaatcttt agaagaagca aggaagattt tcaggtttta |
| 781 | ataacgcagt catggaggcc aagaacttgc aataccaatg ttctaaagtt ttgcaacaat |
| 841 | aaagtacttt acctaagtgt tgattgtgcc tgttgtgaag ctaatgaact ctttcaaatt |
| 901 | atatgctaat taaataatac aactcctatt cgctgactta gttaaaattg atttgttttc |
| 961 | attaggatct gatgtgaatt cagatttcca atcttctcct agccaaccat tttcctggaa |
| 1021 | ttaaaaattc agtaaaaaag gaaactatag attatgtggt ttgtttgact tttccaagaa |
| 1081 | ttgtcccgta acatacaatt tgtcatacaa tctattaaaa tgtcaatgta gaaatgcact |
| 1141 | tctgacattt tcaggtatgc acaggagaag agttaccatc ctggataatg gcataaagac |
| 1201 | attttcttct tttcctggac agtcatttta tttctgataa aagcgttctt tcttatgcaa |
| 1261 | aaaaaaaaaa aaaaaa |

A further object of the present invention relates to a method for determining the survival time of subject suffering from a cancer comprising the steps of i) determining the expression level of hGSTA1 in a tumor sample obtained from the subject, ii) comparing the expression level determined at step i) with its predetermined reference value and ii) concluding that the subject will have a long survival time when the expression level of hGSTA1 is higher than its predetermined reference value or concluding that the subject will have a short survival time when the expression level of hGSTA1 is lower than its predetermined reference value.

The method is particularly suitable for predicting the duration of the overall survival (OS), progression-free survival (PFS) and/or the disease-free survival (DFS) of the cancer subject. Those of skill in the art will recognize that OS survival time is generally based on and expressed as the percentage of people who survive a certain type of cancer for a specific amount of time. Cancer statistics often use an overall five-year survival rate. In general, OS rates do not specify whether cancer survivors are still undergoing treatment at five years or if they've become cancer-free (achieved remission). DSF gives more specific information and is the number of people with a particular cancer who achieve remission. Also, progression-free survival (PFS) rates (the number of people who still have cancer, but their disease does not progress) includes people who may have had some success with treatment, but the cancer has not disappeared completely. As used herein, the expression “short survival time” indicates that the subject will have a survival time that will be lower than the median (or mean) observed in the general population of subjects suffering from said cancer. When the subject will have a short survival time, it is meant that the subject will have a “poor prognosis”. Inversely, the expression “long survival time” indicates that the subject will have a survival time that will be higher than the median (or mean) observed in the general population of subjects suffering from said cancer. When the subject will have a long survival time, it is meant that the subject will have a “good prognosis”.

A further object of the present invention relates to a method for determining whether a subject suffering from a cancer will achieve a response with dendrogenin A of i) determining the expression level of hGSTA1 in a tumor sample obtained from the subject, ii) comparing the expression level determined at step i) with its predetermined reference value and ii) concluding that the subject will achieve a response with dendrogenin A when the expression level of hGSTA1 is lower than its predetermined reference value.

As used herein, the term “Dendrogenin A” refers to the pharmaceutically active compound 5-hydroxy-6-[2-(1H-imidazol-4-yl)ethylamino]cholestan-3-ol. Dendrogenin A is disclosed in WO03/89449 and de Medina et al (J. Med. Chem., 2009) as free base. Its structural formula is the following:

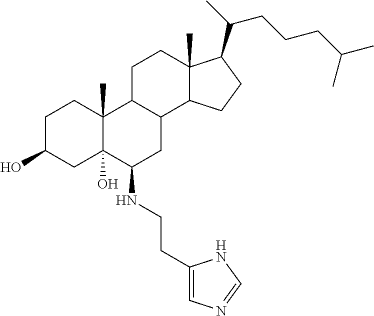 [SMILES: CC(C)CCCC(C)[C@H]1CCC2C3C[C@@H](NCCc4cnc[nH]4)[C@@]4(O)C[C@@H](O)CC[C@]4(C)C3CC[C@@]21C]

Measuring the expression level of a gene (i.e. hGSTA1) can be performed by a variety of techniques well known in the art.

In some embodiments, the expression level is determined at nucleic acid level. Typically, the expression level of a gene may be determined by determining the quantity of mRNA. Methods for determining the quantity of mRNA are well known in the art. For example the nucleic acid contained in the samples (e.g., cell or tissue prepared from the subject) is first extracted according to standard methods, for example using lytic enzymes or chemical solutions or extracted by nucleic-acid-binding resins following the manufacturer's instructions. The extracted mRNA is then detected by hybridization (e. g., Northern blot analysis, in situ hybridization) and/or amplification (e.g., RT-PCR). Other methods of Amplification include ligase chain reaction (LCR), transcription-mediated amplification (TMA), strand displacement amplification (SDA) and nucleic acid sequence based amplification (NASBA).

Nucleic acids having at least 10 nucleotides and exhibiting sequence complementarity or homology to the mRNA of interest herein find utility as hybridization probes or amplification primers. It is understood that such nucleic acids need not be identical, but are typically at least about 80% identical to the homologous region of comparable size, more preferably 85% identical and even more preferably 90-95% identical. In certain embodiments, it will be advantageous to use nucleic acids in combination with appropriate means, such as a detectable label, for detecting hybridization.

Typically, the nucleic acid probes include one or more labels, for example to permit detection of a target nucleic acid molecule using the disclosed probes. In various applications, such as in situ hybridization procedures, a nucleic acid probe includes a label (e.g., a detectable label). A “detectable label” is a molecule or material that can be used to produce a detectable signal that indicates the presence or concentration of the probe (particularly the bound or hybridized probe) in a sample. Thus, a labeled nucleic acid molecule provides an indicator of the presence or concentration of a target nucleic acid sequence (e.g., genomic target nucleic acid sequence) (to which the labeled uniquely specific nucleic acid molecule is bound or hybridized) in a sample. A label associated with one or more nucleic acid molecules (such as a probe generated by the disclosed methods) can be detected either directly or indirectly. A label can be detected by any known or yet to be discovered mechanism including absorption, emission and/or scattering of a photon (including radio frequency, microwave frequency, infrared frequency, visible frequency and ultra-violet frequency photons). Detectable labels include colored, fluorescent, phosphorescent and luminescent molecules and materials, catalysts (such as enzymes) that convert one substance into another substance to provide a detectable difference (such as by converting a colorless substance into a colored substance or vice versa, or by producing a precipitate or increasing sample turbidity), haptens that can be detected by antibody binding interactions, and paramagnetic and magnetic molecules or materials.

Particular examples of detectable labels include fluorescent molecules (or fluorochromes). Numerous fluorochromes are known to those of skill in the art, and can be selected, for example from Life Technologies (formerly Invitrogen), e.g., see, The Handbook—A Guide to Fluorescent Probes and Labeling Technologies). Examples of particular fluorophores that can be attached (for example, chemically conjugated) to a nucleic acid molecule (such as a uniquely specific binding region) are provided in U.S. Pat. No. 5,866,366 to Nazarenko et al., such as 4-acetamido-4′-isothiocyanatostilbene-2,2′ disulfonic acid, acridine and derivatives such as acridine and acridine isothiocyanate, 5-(2′-aminoethyl) aminonaphthalene-1-sulfonic acid (EDANS), 4-amino-N-[3 vinylsulfonyl)phenyl]naphthalimide-3,5 disulfonate (Lucifer Yellow VS), N-(4-anilino-1-naphthyl)maleimide, antllranilamide, Brilliant Yellow, coumarin and derivatives such as coumarin, 7-amino-4-methylcoumarin (AMC, Coumarin 120), 7-amino-4-trifluoromethylcouluarin (Coumarin 151); cyanosine; 4′,6-diarninidino-2-phenylindole (DAPI); 5′,5″dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulforlic acid; 5-[dimethylamino] naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6dicl1lorotriazin-2-yDarninofluorescein (DTAF), 2′7′dimethoxy-4′5′-dichloro-6-carboxyfluorescein (JOE), fluorescein, fluorescein isothiocyanate (FITC), and QFITC Q(RITC); 2′,7′-difluorofluorescein (OREGON GREEN®); fluorescamine; IR144; IR1446; Malachite Green isothiocyanate; 4-methylumbelliferone; ortho cresolphthalein; nitrotyrosine; pararosaniline; Phenol Red; B-phycoerythrin; o-phthaldialdehyde; pyrene and derivatives such as pyrene, pyrene butyrate and succinimidyl 1-pyrene butyrate; Reactive Red 4 (Cibacron Brilliant Red 3B-A); rhodamine and derivatives such as 6-carboxy-X-rhodamine (ROX), 6-carboxyrhodamine (R6G), lissamine rhodamine B sulfonyl chloride, rhodamine (Rhod), rhodamine B, rhodamine 123, rhodamine X isothiocyanate, rhodamine green, sulforhodamine B, sulforhodamine 101 and sulfonyl chloride derivative of sulforhodamine 101 (Texas Red); N,N,N′,N′-tetramethyl-6-carboxyrhodamine (TAMRA); tetramethyl rhodamine; tetramethyl rhodamine isothiocyanate (TRITC); riboflavin; rosolic acid and terbium chelate derivatives. Other suitable fluorophores include thiol-reactive europium chelates which emit at approximately 617 mn (Heyduk and Heyduk, Analyt. Biochem. 248:216-27, 1997; J. Biol. Chem. 274:3315-22, 1999), as well as GFP, Lissamine™, diethylaminocoumarin, fluorescein chlorotriazinyl, naphthofluorescein, 4,7-dichlororhodamine and xanthene (as described in U.S. Pat. No. 5,800,996 to Lee et al.) and derivatives thereof. Other fluorophores known to those skilled in the art can also be used, for example those available from Life Technologies (Invitrogen; Molecular Probes (Eugene, Oreg.)) and including the ALEXA FLUOR® series of dyes (for example, as described in U.S. Pat. Nos. 5,696,157, 6,130,101 and 6,716,979), the BODIPY series of dyes (dipyrrometheneboron difluoride dyes, for example as described in U.S. Pat. Nos. 4,774,339, 5,187,288, 5,248,782, 5,274,113, 5,338,854, 5,451,663 and 5,433,896), Cascade Blue (an amine reactive derivative of the sulfonated pyrene described in U.S. Pat. No. 5,132,432) and Marina Blue (U.S. Pat. No. 5,830,912).

In addition to the fluorochromes described above, a fluorescent label can be a fluorescent nanoparticle, such as a semiconductor nanocrystal, e.g., a QUANTUM DOT™ (obtained, for example, from Life Technologies (QuantumDot Corp, Invitrogen Nanocrystal Technologies, Eugene, Oreg.); see also, U.S. Pat. Nos. 6,815,064; 6,682,596; and 6,649, 138). Semiconductor nanocrystals are microscopic particles having size-dependent optical and/or electrical properties. When semiconductor nanocrystals are illuminated with a primary energy source, a secondary emission of energy occurs of a frequency that corresponds to the handgap of the semiconductor material used in the semiconductor nanocrystal. This emission can he detected as colored light of a specific wavelength or fluorescence. Semiconductor nanocrystals with different spectral characteristics are described in e.g., U.S. Pat. No. 6,602,671. Semiconductor nanocrystals that can he coupled to a variety of biological molecules (including dNTPs and/or nucleic acids) or substrates by techniques described in, for example, Bruchez et al., Science 281:20132016, 1998; Chan et al., Science 281:2016-2018, 1998; and U.S. Pat. No. 6,274,323. Formation of semiconductor nanocrystals of various compositions are disclosed in, e.g., U.S. Pat. Nos. 6,927,069; 6,914,256; 6,855,202; 6,709,929; 6,689,338; 6,500,622; 6,306,736; 6,225,198; 6,207,392; 6,114,038; 6,048,616; 5,990,479; 5,690,807; 5,571,018; 5,505,928; 5,262,357 and in U.S. Patent Publication No. 2003/0165951 as well as PCT Publication No. 99/26299 (puhlished May 27, 1999). Separate populations of semiconductor nanocrystals can he produced that are identifiable based on their different spectral characteristics. For example, semiconductor nanocrystals can he produced that emit light of different colors hased on their composition, size or size and composition. For example, quantum dots that emit light at different wavelengths based on size (565 mn, 655 mn, 705 mn, or 800 mn emission wavelengths), which are suitable as fluorescent labels in the probes disclosed herein are available from Life Technologies (Carlshad, Calif.).

Additional labels include, for example, radioisotopes (such as ³H), metal chelates such as DOTA and DPTA chelates of radioactive or paramagnetic metal ions like Gd3+, and liposomes.

Detectable labels that can he used with nucleic acid molecules also include enzymes, for example horseradish peroxidase, alkaline phosphatase, acid phosphatase, glucose oxidase, beta-galactosidase, beta-glucuronidase, or beta-lactamase.

Alternatively, an enzyme can he used in a metallographic detection scheme. For example, silver in situ hyhridization (SISH) procedures involve metallographic detection schemes for identification and localization of a hybridized genomic target nucleic acid sequence. Metallographic detection methods include using an enzyme, such as alkaline phosphatase, in combination with a water-soluble metal ion and a redox-inactive substrate of the enzyme. The substrate is converted to a redox-active agent by the enzyme, and the redoxactive agent reduces the metal ion, causing it to form a detectable precipitate. (See, for example, U.S. Patent Application Publication No. 2005/0100976, PCT Publication No. 2005/003777 and U.S. Patent Application Publication No. 2004/0265922). Metallographic detection methods also include using an oxido-reductase enzyme (such as horseradish peroxidase) along with a water soluble metal ion, an oxidizing agent and a reducing agent, again to form a detectable precipitate. (See, for example, U.S. Pat. No. 6,670,113).

Probes made using the disclosed methods can be used for nucleic acid detection, such as ISH procedures (for example, fluorescence in situ hybridization (FISH), chromogenic in situ hybridization (CISH) and silver in situ hybridization (SISH)) or comparative genomic hybridization (CGH).

In situ hybridization (ISH) involves contacting a sample containing target nucleic acid sequence (e.g., genomic target nucleic acid sequence) in the context of a metaphase or interphase chromosome preparation (such as a cell or tissue sample mounted on a slide) with a labeled probe specifically hybridizable or specific for the target nucleic acid sequence (e.g., genomic target nucleic acid sequence). The slides are optionally pretreated, e.g., to remove paraffin or other materials that can interfere with uniform hybridization. The sample and the probe are both treated, for example by heating to denature the double stranded nucleic acids. The probe (formulated in a suitable hybridization buffer) and the sample are combined, under conditions and for sufficient time to permit hybridization to occur (typically to reach equilibrium). The chromosome preparation is washed to remove excess probe, and detection of specific labeling of the chromosome target is performed using standard techniques.

For example, a biotinylated probe can be detected using fluorescein-labeled avidin or avidin-alkaline phosphatase. For fluorochrome detection, the fluorochrome can be detected directly, or the samples can be incubated, for example, with fluorescein isothiocyanate (FITC)-conjugated avidin. Amplification of the FITC signal can be effected, if necessary, by incubation with biotin-conjugated goat antiavidin antibodies, washing and a second incubation with FITC-conjugated avidin. For detection by enzyme activity, samples can be incubated, for example, with streptavidin, washed, incubated with biotin-conjugated alkaline phosphatase, washed again and pre-equilibrated (e.g., in alkaline phosphatase (AP) buffer). For a general description of in situ hybridization procedures, see, e.g., U.S. Pat. No. 4,888,278.

Numerous procedures for FISH, CISH, and SISH are known in the art. For example, procedures for performing FISH are described in U.S. Pat. Nos. 5,447,841; 5,472,842; and 5,427,932; and for example, in Pirlkel et al., Proc. Natl. Acad. Sci. 83:2934-2938, 1986; Pinkel et al., Proc. Natl. Acad. Sci. 85:9138-9142, 1988; and Lichter et al., Proc. Natl. Acad. Sci. 85:9664-9668, 1988. CISH is described in, e.g., Tanner et al., Am. .1. Pathol. 157:1467-1472, 2000 and U.S. Pat. No. 6,942,970. Additional detection methods are provided in U.S. Pat. No. 6,280,929.

Numerous reagents and detection schemes can be employed in conjunction with FISH, CISH, and SISH procedures to improve sensitivity, resolution, or other desirable properties. As discussed above probes labeled with fluorophores (including fluorescent dyes and QUANTUM DOTS®) can be directly optically detected when performing FISH. Alternatively, the probe can be labeled with a nonfluorescent molecule, such as a hapten (such as the following non-limiting examples: biotin, digoxigenin, DNP, and various oxazoles, pyrrazoles, thiazoles, nitroaryls, benzofurazans, triterpenes, ureas, thioureas, rotenones, coumarin, courmarin-based compounds, Podophyllotoxin, Podophyllotoxin-based compounds, and combinations thereof), ligand or other indirectly detectable moiety. Probes labeled with such non-fluorescent molecules (and the target nucleic acid sequences to which they bind) can then be detected by contacting the sample (e.g., the cell or tissue sample to which the probe is bound) with a labeled detection reagent, such as an antibody (or receptor, or other specific binding partner) specific for the chosen hapten or ligand. The detection reagent can be labeled with a fluorophore (e.g., QUANTUM DOT®) or with another indirectly detectable moiety, or can be contacted with one or more additional specific binding agents (e.g., secondary or specific antibodies), which can be labeled with a fluorophore.

In other examples, the probe, or specific binding agent (such as an antibody, e.g., a primary antibody, receptor or other binding agent) is labeled with an enzyme that is capable of converting a fluorogenic or chromogenic composition into a detectable fluorescent, colored or otherwise detectable signal (e.g., as in deposition of detectable metal particles in SISH). As indicated above, the enzyme can be attached directly or indirectly via a linker to the relevant probe or detection reagent. Examples of suitable reagents (e.g., binding reagents) and chemistries (e.g., linker and attachment chemistries) are described in U.S. Patent Application Publication Nos. 2006/0246524; 2006/0246523, and 2007/01 17153.

It will he appreciated by those of skill in the art that by appropriately selecting labelled probe-specific binding agent pairs, multiplex detection schemes can he produced to facilitate detection of multiple target nucleic acid sequences (e.g., genomic target nucleic acid sequences) in a single assay (e.g., on a single cell or tissue sample or on more than one cell or tissue sample). For example, a first probe that corresponds to a first target sequence can he labelled with a first hapten, such as biotin, while a second probe that corresponds to a second target sequence can be labelled with a second hapten, such as DNP. Following exposure of the sample to the probes, the bound probes can he detected by contacting the sample with a first specific binding agent (in this case avidin labelled with a first fluorophore, for example, a first spectrally distinct QUANTUM DOT®, e.g., that emits at 585 mn) and a second specific binding agent (in this case an anti-DNP antibody, or antibody fragment, labelled with a second fluorophore (for example, a second spectrally distinct QUANTUM DOT®, e.g., that emits at 705 mn). Additional probes/binding agent pairs can he added to the multiplex detection scheme using other spectrally distinct fluorophores. Numerous variations of direct, and indirect (one step, two step or more) can he envisioned, all of which are suitable in the context of the disclosed probes and assays.

Probes typically comprise single-stranded nucleic acids of between 10 to 1000 nucleotides in length, for instance of between 10 and 800, more preferably of between 15 and 700, typically of between 20 and 500. Primers typically are shorter single-stranded nucleic acids, of between 10 to 25 nucleotides in length, designed to perfectly or almost perfectly match a nucleic acid of interest, to be amplified. The probes and primers are “specific” to the nucleic acids they hybridize to, i.e. they preferably hybridize under high stringency hybridization conditions (corresponding to the highest melting temperature Tm, e.g., 50% formamide, 5× or 6×SCC. SCC is a 0.15 M NaCl, 0.015 M Na-citrate).

The nucleic acid primers or probes used in the above amplification and detection method may be assembled as a kit. Such a kit includes consensus primers and molecular probes. A preferred kit also includes the components necessary to determine if amplification has occurred. The kit may also include, for example, PCR buffers and enzymes; positive control sequences, reaction control primers; and instructions for amplifying and detecting the specific sequences.

In some embodiments, the methods of the invention comprise the steps of providing total RNAs extracted from cumulus cells and subjecting the RNAs to amplification and hybridization to specific probes, more particularly by means of a quantitative or semi-quantitative RT-PCR.

In some embodiments, the expression level is determined by DNA chip analysis. Such DNA chip or nucleic acid microarray consists of different nucleic acid probes that are chemically attached to a substrate, which can be a microchip, a glass slide or a microsphere-sized bead. A microchip may be constituted of polymers, plastics, resins, polysaccharides, silica or silica-based materials, carbon, metals, inorganic glasses, or nitrocellulose. Probes comprise nucleic acids such as cDNAs or oligonucleotides that may be about 10 to about 60 base pairs. To determine the expression level, a sample from a test subject, optionally first subjected to a reverse transcription, is labelled and contacted with the microarray in hybridization conditions, leading to the formation of complexes between target nucleic acids that are complementary to probe sequences attached to the microarray surface. The labelled hybridized complexes are then detected and can be quantified or semi-quantified. Labelling may be achieved by various methods, e.g. by using radioactive or fluorescent labelling. Many variants of the microarray hybridization technology are available to the man skilled in the art (see e.g. the review by Hoheisel, Nature Reviews, Genetics, 2006, 7:200-210).

In some embodiments, the nCounter® Analysis system is used to detect intrinsic gene expression. The basis of the nCounter® Analysis system is the unique code assigned to each nucleic acid target to be assayed (International Patent Application Publication No. WO 08/124847, U.S. Pat. No. 8,415,102 and Geiss et al. Nature Biotechnology. 2008. 26(3): 317-325; the contents of which are each incorporated herein by reference in their entireties). The code is composed of an ordered series of colored fluorescent spots which create a unique barcode for each target to be assayed. A pair of probes is designed for each DNA or RNA target, a biotinylated capture probe and a reporter probe carrying the fluorescent barcode. This system is also referred to, herein, as the nanoreporter code system. Specific reporter and capture probes are synthesized for each target. The reporter probe can comprise at a least a first label attachment region to which are attached one or more label monomers that emit light constituting a first signal; at least a second label attachment region, which is non-over-lapping with the first label attachment region, to which are attached one or more label monomers that emit light constituting a second signal; and a first target-specific sequence. Preferably, each sequence specific reporter probe comprises a target specific sequence capable of hybridizing to no more than one gene and optionally comprises at least three, or at least four label attachment regions, said attachment regions comprising one or more label monomers that emit light, constituting at least a third signal, or at least a fourth signal, respectively. The capture probe can comprise a second target-specific sequence; and a first affinity tag. In some embodiments, the capture probe can also comprise one or more label attachment regions. Preferably, the first target-specific sequence of the reporter probe and the second target-specific sequence of the capture probe hybridize to different regions of the same gene to be detected. Reporter and capture probes are all pooled into a single hybridization mixture, the “probe library”. The relative abundance of each target is measured in a single multiplexed hybridization reaction. The method comprises contacting the tumor sample with a probe library, such that the presence of the target in the sample creates a probe pair—target complex. The complex is then purified. More specifically, the sample is combined with the probe library, and hybridization occurs in solution. After hybridization, the tripartite hybridized complexes (probe pairs and target) are purified in a two-step procedure using magnetic beads linked to oligonucleotides complementary to universal sequences present on the capture and reporter probes. This dual purification process allows the hybridization reaction to be driven to completion with a large excess of target-specific probes, as they are ultimately removed, and, thus, do not interfere with binding and imaging of the sample. All post hybridization steps are handled robotically on a custom liquid-handling robot (Prep Station, NanoString Technologies). Purified reactions are typically deposited by the Prep Station into individual flow cells of a sample cartridge, bound to a streptavidin-coated surface via the capture probe, electrophoresed to elongate the reporter probes, and immobilized. After processing, the sample cartridge is transferred to a fully automated imaging and data collection device (Digital Analyzer, NanoString Technologies). The expression level of a target is measured by imaging each sample and counting the number of times the code for that target is detected. For each sample, typically 600 fields-of-view (FOV) are imaged (1376×1024 pixels) representing approximately 10 mm2 of the binding surface. Typical imaging density is 100-1200 counted reporters per field of view depending on the degree of multiplexing, the amount of sample input, and overall target abundance. Data is output in simple spreadsheet format listing the number of counts per target, per sample. This system can be used along with nanoreporters. Additional disclosure regarding nanoreporters can be found in International Publication No. WO 07/076129 and WO07/076132, and US Patent Publication No. 2010/0015607 and 2010/0261026, the contents of which are incorporated herein in their entireties. Further, the term nucleic acid probes and nanoreporters can include the rationally designed (e.g. synthetic sequences) described in International Publication No. WO 2010/019826 and US Patent Publication No. 2010/0047924, incorporated herein by reference in its entirety.

Expression level of a gene may be expressed as absolute expression level or normalized expression level. Typically, expression levels are normalized by correcting the absolute expression level of a gene by comparing its expression to the expression of a gene that is not a relevant for determining the cancer stage of the subject, e.g., a housekeeping gene that is constitutively expressed. Suitable genes for normalization include housekeeping genes such as the actin gene ACTB, ribosomal 18S gene, GUSB, PGK1 and TFRC. This normalization allows the comparison of the expression level in one sample, e.g., a subject sample, to another sample, or between samples from different sources.

In some embodiments, the expression level of hGSTA1 is determined at the protein level by any well known method in the art. Typically, such methods comprise contacting the tumor tissue sample with at least one selective binding agent capable of selectively interacting with hGSTA1. The selective binding agent may be polyclonal antibody or monoclonal antibody, an antibody fragment, synthetic antibodies, or other protein-specific agents such as nucleic acid or peptide aptamers. Several antibodies have been described in the prior art and many antibodies are also commercially available such as described in the EXAMPLE. For the detection of the antibody that makes the presence of the hGSTA1 detectable by microscopy or an automated analysis system, the antibodies may be tagged directly with detectable labels such as enzymes, chromogens or fluorescent probes or indirectly detected with a secondary antibody conjugated with detectable labels. The preferred method according to the present invention is immunohistochemistry. Immunohistochemistry typically includes the following steps:
fixing said tumor sample with formalin,
embedding said tumor sample in paraffin.
cutting said tumor sample into sections for staining
incubating said sections with the binding partner specific for
rinsing said sections
incubating said section with a biotinylated secondary antibody
revealing the antigen-antibody complex with avidin-biotin-peroxidase complex
   Accordingly, the tissue tumor sample is firstly incubated the binding partners. After washing, the labeled antibodies that are bound to marker of interest are revealed by the appropriate technique, depending of the kind of label is borne by the labeled antibody, e.g. radioactive, fluorescent or enzyme label. Multiple labelling can be performed simultaneously. Alternatively, the method of the present invention may use a secondary antibody coupled to an amplification system (to intensify staining signal) and enzymatic molecules. Such coupled secondary antibodies are commercially available, e.g. from Dako, EnVision system. Counterstaining may be used, e.g. H&E, DAPI, Hoechst. Other staining methods may be accomplished using any suitable method or system as would be apparent to one of skill in the art, including automated, semi-automated or manual systems.

Typically, the predetermined reference value is a threshold value or a cut-off value. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. For example, retrospective measurement of expression level of hGSTA1 in properly banked historical subject samples may be used in establishing the predetermined reference value. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. For example, after determining the expression level of hGSTA1 in a group of reference, one can use algorithmic analysis for the statistic treatment of the measured expression levels of hGSTA1 in samples to be tested, and thus obtain a classification standard having significance for sample classification. The full name of ROC curve is receiver operator characteristic curve, which is also known as receiver operation characteristic curve. It is mainly used for clinical biochemical diagnostic tests. ROC curve is a comprehensive indicator that reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is quite high. This algorithmic method is preferably done with a computer. Existing software or systems in the art may be used for the drawing of the ROC curve, such as: MedCalc 9.2.0.1 medical statistical software, SPSS 9.0, ROCPOWER.SAS, DESIGNROC.FOR, MULTIREADER POWER.SAS, CREATE-ROC.SAS, GB STAT VI0.0 (Dynamic Microsystems, Inc. Silver Spring, Md., USA), etc.

A predetermined reference value can be relative to a number or value derived from population studies, including without limitation, subjects of the same or similar age range, subjects in the same or similar ethnic group, and subjects having the same severity of cancer. Such predetermined reference values can be derived from statistical analyses and/or risk prediction data of populations obtained from mathematical algorithms and computed indices. In some embodiments, the predetermined reference values are derived from the expression level of hGSTA1 in a control sample derived from one or more subjects who do not suffer from cancer. Furthermore, retrospective measurement of the expression level of hGSTA1 in properly banked historical subject samples may be used in establishing these predetermined reference values.

In some embodiments, the predetermined reference value is the expression level of hGSTA1 determined in the adjacent non tumoral tissue.

In some embodiments, the predetermined reference value is correlated the survival time (e.g. disease-free survival (DFS) and/or the overall survival (OS)). Accordingly, the predetermined reference value may be typically determined by carrying out a method comprising the steps of

a) providing a collection of tumor samples from subject suffering from the same cancer;

b) providing, for each tumor sample provided at step a), information relating to the actual clinical outcome for the corresponding subject (i.e. the duration of the disease-free survival (DFS) and/or the overall survival (OS));

c) providing a serial of arbitrary quantification values;

d) determining the level of hGSTA1 for each tumor sample contained in the collection provided at step a);

e) classifying said tumor samples in two groups for one specific arbitrary quantification value provided at step c), respectively: (i) a first group comprising tumor samples that exhibit a quantification value for level that is lower than the said arbitrary quantification value contained in the said serial of quantification values; (ii) a second group comprising tumor samples that exhibit a quantification value for said level that is higher than the said arbitrary quantification value contained in the said serial of quantification values; whereby two groups of tumor samples are obtained for the said specific quantification value, wherein the tumor samples of each group are separately enumerated;

f) calculating the statistical significance between (i) the quantification value obtained at step e) and (ii) the actual clinical outcome of the subjects from which tumor samples contained in the first and second groups defined at step f) derive;

g) reiterating steps f) and g) until every arbitrary quantification value provided at step d) is tested;

h) setting the said predetermined reference value as consisting of the arbitrary quantification value for which the highest statistical significance (most significant) has been calculated at step g).

For example the expression level of hGSTA1 has been assessed for 100 tumor samples of 100 subjects. The 100 samples are ranked according to the expression level of hGSTA1. Sample 1 has the highest level and sample 100 has the lowest level. A first grouping provides two subsets: on one side sample Nr 1 and on the other side the 99 other samples. The next grouping provides on one side samples 1 and 2 and on the other side the 98 remaining samples etc., until the last grouping: on one side samples 1 to 99 and on the other side sample Nr 100. According to the information relating to the actual clinical outcome for the corresponding cancer subject, Kaplan Meier curves are prepared for each of the 99 groups of two subsets. Also for each of the 99 groups, the p value between both subsets was calculated. The predetermined reference value is then selected such as the discrimination based on the criterion of the minimum p value is the strongest. In other terms, the expression level of hGSTA1 corresponding to the boundary between both subsets for which the p value is minimum is considered as the predetermined reference value. It should be noted that the predetermined reference value is not necessarily the median value of levels of hGSTA1.

Thus in some embodiments, the predetermined reference value thus allows discrimination between a poor and a good prognosis with respect to DFS and OS for a subject. Practically, high statistical significance values (e.g. low P values) are generally obtained for a range of successive arbitrary quantification values, and not only for a single arbitrary quantification value. Thus, in one alternative embodiment of the invention, instead of using a definite predetermined reference value, a range of values is provided. Therefore, a minimal statistical significance value (minimal threshold of significance, e.g. maximal threshold P value) is arbitrarily set and a range of a plurality of arbitrary quantification values for which the statistical significance value calculated at step g) is higher (more significant, e.g. lower P value) are retained, so that a range of quantification values is provided. This range of quantification values includes a “cut-off” value as described above.

For example, according to this specific embodiment of a “cut-off” value, the outcome can be determined by comparing the expression level of hGSTA1 with the range of values which are identified. In certain embodiments, a cut-off value thus consists of a range of quantification values, e.g. centered on the quantification value for which the highest statistical significance value is found (e.g. generally the minimum p value which is found). For example, on a hypothetical scale of 1 to 10, if the ideal cut-off value (the value with the highest statistical significance) is 5, a suitable (exemplary) range may be from 4-6. For example, a subject may be assessed by comparing values obtained by measuring the expression level of hGSTA1, where values greater than 5 reveal a good prognosis and values less than 5 reveal a poor prognosis. In a another embodiment, a subject may be assessed by comparing values obtained by measuring the expression level of hGSTA1 and comparing the values on a scale, where values above the range of 4-6 indicate a good prognosis and values below the range of 4-6 indicate a poor prognosis, with values falling within the range of 4-6 indicating an intermediate occurrence (or prognosis).

### Therapeutic Methods of the Invention

Once the subject is diagnosed as suffering from cancer, the physician can take the choice to administer the subject with the most accurate treatment. Typically, the treatment includes chemotherapy, radiotherapy, and immunotherapy.

In some embodiments, the subject once diagnosed as suffering from cancer by the method of the invention is administered with a chemotherapeutic agent. The term “chemotherapeutic agent” refers to chemical compounds that are effective in inhibiting tumor growth. Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaorarnide and trimethylolomelamine; acetogenins (especially bullatacin and bullatacinone); a carnptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estrarnustine, ifosfamide, mechlorethamine, mechlorethamine oxide hydrochloride, melphalan, novembichin, phenesterine, prednimus tine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin (11 and calicheamicin 211, see, e.g., Agnew Chem Intl. Ed. Engl. 33:183-186 (1994); dynemicin, including dynemicin A; an esperamicin; as well as neocarzinostatin chromophore and related chromoprotein enediyne antiobiotic chromomophores), aclacinomysins, actinomycin, authramycin, azaserine, bleomycin, cactinomycin, carabicin, canninomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idanrbicin, marcellomycin, mitomycins, mycophenolic acid, nogalarnycin, olivomycins, peplomycin, potfiromycin, puromycin, quelamycin, rodorubicin, streptomgrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmo fur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophospharnide glycoside; amino levulinic acid; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pento statin; phenamet; pirarubicin; podophyllinic acid; 2-ethylhydrazide; procarbazine; PSK®; razoxane; rhizoxin; sizofiran; spirogennanium; tenuazonic acid; triaziquone; 2,2′,2″-trichlorotriethylarnine; trichothecenes (especially T-2 toxin, verracurin A, roridinA and anguidine); urethan; vindesine; dacarbazine; mannomustine; mitobromtol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.].) and doxetaxel (TAXOTERE®, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-1 1; topoisomerase inhibitor RFS 2000; difluoromethylornithine (DMFO); retinoic acid; capecitabine; and phannaceutically acceptable salts, acids or derivatives of any of the above. Also included in this definition are antihormonal agents that act to regulate or inhibit honnone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and phannaceutically acceptable salts, acids or derivatives of any of the above.

In some embodiments, the subject once diagnosed as suffering from cancer is administered with a targeted cancer therapy. Targeted cancer therapies are drugs or other substances that block the growth and spread of cancer by interfering with specific molecules (“molecular targets”) that are involved in the growth, progression, and spread of cancer. Targeted cancer therapies are sometimes called “molecularly targeted drugs,” “molecularly targeted therapies,” “precision medicines,” or similar names. In some embodiments, the targeted therapy consists of administering the subject with a tyrosine kinase inhibitor. The term “tyrosine kinase inhibitor” refers to any of a variety of therapeutic agents or drugs that act as selective or non-selective inhibitors of receptor and/or non-receptor tyrosine kinases. Tyrosine kinase inhibitors and related compounds are well known in the art and described in U.S Patent Publication 2007/0254295, which is incorporated by reference herein in its entirety. It will be appreciated by one of skill in the art that a compound related to a tyrosine kinase inhibitor will recapitulate the effect of the tyrosine kinase inhibitor, e.g., the related compound will act on a different member of the tyrosine kinase signaling pathway to produce the same effect as would a tyrosine kinase inhibitor of that tyrosine kinase. Examples of tyrosine kinase inhibitors and related compounds suitable for use in methods of embodiments of the present invention include, but are not limited to, dasatinib (BMS-354825), PP2, BEZ235, saracatinib, gefitinib (Iressa), sunitinib (Sutent; SU11248), erlotinib (Tarceva; OSI-1774), lapatinib (GW572016; GW2016), canertinib (CI 1033), semaxinib (SU5416), vatalanib (PTK787/ZK222584), sorafenib (BAY 43-9006), imatinib (Gleevec; STI571), leflunomide (SU101), vandetanib (Zactima; ZD6474), MK-2206 (8-[4-aminocyclobutyl)phenyl]-9-phenyl-1,2,4-triazolo [3,4-f] [1,6]naphthyridin-3 (2H)-one hydrochloride) derivatives thereof, analogs thereof, and combinations thereof. Additional tyrosine kinase inhibitors and related compounds suitable for use in the present invention are described in, for example, U.S Patent Publication 2007/0254295, U.S. Pat. Nos. 5,618,829, 5,639,757, 5,728,868, 5,804,396, 6,100,254, 6,127,374, 6,245,759, 6,306,874, 6,313,138, 6,316,444, 6,329,380, 6,344,459, 6,420,382, 6,479,512, 6,498,165, 6,544,988, 6,562,818, 6,586,423, 6,586,424, 6,740,665, 6,794,393, 6,875,767, 6,927,293, and 6,958,340, all of which are incorporated by reference herein in their entirety. In certain embodiments, the tyrosine kinase inhibitor is a small molecule kinase inhibitor that has been orally administered and that has been the subject of at least one Phase I clinical trial, more preferably at least one Phase II clinical, even more preferably at least one Phase III clinical trial, and most preferably approved by the FDA for at least one hematological or oncological indication. Examples of such inhibitors include, but are not limited to, Gefitinib, Erlotinib, Lapatinib, Canertinib, BMS-599626 (AC-480), Neratinib, KRN-633, CEP-11981, Imatinib, Nilotinib, Dasatinib, AZM-475271, CP-724714, TAK-165, Sunitinib, Vatalanib, CP-547632, Vandetanib, Bosutinib, Lestaurtinib, Tandutinib, Midostaurin, Enzastaurin, AEE-788, Pazopanib, Axitinib, Motasenib, OSI-930, Cediranib, KRN-951, Dovitinib, Seliciclib, SNS-032, PD-0332991, MKC-I (Ro-317453; R-440), Sorafenib, ABT-869, Brivanib (BMS-582664), SU-14813, Telatinib, SU-6668, (TSU-68), L-21649, MLN-8054, AEW-541, and PD-0325901.

In some embodiments, the subject once diagnosed as suffering from a cancer is administered with an immunotherapeutic agent. The term “immunotherapeutic agent,” as used herein, refers to a compound, composition or treatment that indirectly or directly enhances, stimulates or increases the body's immune response against cancer cells and/or that decreases the side effects of other anticancer therapies. Immunotherapy is thus a therapy that directly or indirectly stimulates or enhances the immune system's responses to cancer cells and/or lessens the side effects that may have been caused by other anti-cancer agents. Immunotherapy is also referred to in the art as immunologic therapy, biological therapy biological response modifier therapy and biotherapy. Examples of common immunotherapeutic agents known in the art include, but are not limited to, cytokines, cancer vaccines, monoclonal antibodies and non-cytokine adjuvants. Alternatively the immunotherapeutic treatment may consist of administering the subject with an amount of immune cells (T cells, NK, cells, dendritic cells, B cells . . . ).

Immunotherapeutic agents can be non-specific, i.e. boost the immune system generally so that the human body becomes more effective in fighting the growth and/or spread of cancer cells, or they can be specific, i.e. targeted to the cancer cells themselves immunotherapy regimens may combine the use of non-specific and specific immunotherapeutic agents.

Non-specific immunotherapeutic agents are substances that stimulate or indirectly improve the immune system. Non-specific immunotherapeutic agents have been used alone as a main therapy for the treatment of cancer, as well as in addition to a main therapy, in which case the non-specific immunotherapeutic agent functions as an adjuvant to enhance the effectiveness of other therapies (e.g. cancer vaccines). Non-specific immunotherapeutic agents can also function in this latter context to reduce the side effects of other therapies, for example, bone marrow suppression induced by certain chemotherapeutic agents. Non-specific immunotherapeutic agents can act on key immune system cells and cause secondary responses, such as increased production of cytokines and immunoglobulins. Alternatively, the agents can themselves comprise cytokines. Non-specific immunotherapeutic agents are generally classified as cytokines or non-cytokine adjuvants.

A number of cytokines have found application in the treatment of cancer either as general non-specific immunotherapies designed to boost the immune system, or as adjuvants provided with other therapies. Suitable cytokines include, but are not limited to, interferons, interleukins and colony-stimulating factors.

Interferons (IFNs) contemplated by the present invention include the common types of IFNs, IFN-alpha (IFN-α), IFN-beta (IFN-β) and IFN-gamma (IFN-γ). IFNs can act directly on cancer cells, for example, by slowing their growth, promoting their development into cells with more normal behaviour and/or increasing their production of antigens thus making the cancer cells easier for the immune system to recognise and destroy. IFNs can also act indirectly on cancer cells, for example, by slowing down angiogenesis, boosting the immune system and/or stimulating natural killer (NK) cells, T cells and macrophages. Recombinant IFN-alpha is available commercially as Roferon (Roche Pharmaceuticals) and Intron A (Schering Corporation).

Interleukins contemplated by the present invention include IL-2, IL-4, IL-11 and IL-12. Examples of commercially available recombinant interleukins include Proleukin® (IL-2; Chiron Corporation) and Neumega® (IL-12; Wyeth Pharmaceuticals). Zymogenetics, Inc. (Seattle, Wash.) is currently testing a recombinant form of IL-21, which is also contemplated for use in the combinations of the present invention.

Colony-stimulating factors (CSFs) contemplated by the present invention include granulocyte colony stimulating factor (G-CSF or filgrastim), granulocyte-macrophage colony stimulating factor (GM-CSF or sargramostim) and erythropoietin (epoetin alfa, darbepoietin). Treatment with one or more growth factors can help to stimulate the generation of new blood cells in subjects undergoing traditional chemotherapy. Accordingly, treatment with CSFs can be helpful in decreasing the side effects associated with chemotherapy and can allow for higher doses of chemotherapeutic agents to be used. Various-recombinant colony stimulating factors are available commercially, for example, Neupogen® (G-CSF; Amgen), Neulasta (pelfilgrastim; Amgen), Leukine (GM-CSF; Berlex), Procrit (erythropoietin; Ortho Biotech), Epogen (erythropoietin; Amgen), Arnesp (erytropoietin).

In addition to having specific or non-specific targets, immunotherapeutic agents can be active, i.e. stimulate the body's own immune response, or they can be passive, i.e. comprise immune system components that were generated external to the body.

Passive specific immunotherapy typically involves the use of one or more monoclonal antibodies that are specific for a particular antigen found on the surface of a cancer cell or that are specific for a particular cell growth factor. Monoclonal antibodies may be used in the treatment of cancer in a number of ways, for example, to enhance a subject's immune response to a specific type of cancer, to interfere with the growth of cancer cells by targeting specific cell growth factors, such as those involved in angiogenesis, or by enhancing the delivery of other anticancer agents to cancer cells when linked or conjugated to agents such as chemotherapeutic agents, radioactive particles or toxins.

Monoclonal antibodies currently used as cancer immunotherapeutic agents that are suitable for inclusion in the combinations of the present invention include, but are not limited to, rituximab (Rituxan®), trastuzumab (Herceptin®), ibritumomab tiuxetan (Zevalin®), tositumomab (Bexxar®), cetuximab (C-225, Erbitux®), bevacizumab (Avastin®), gemtuzumab ozogamicin (Mylotarg®), alemtuzumab (Campath®), and BL22. Other examples include anti-CTLA4 antibodies (e.g. Ipilimumab), anti-PD1 antibodies, anti-PDLL antibodies, anti-TIMP3 antibodies, anti-LAG3 antibodies, anti-B7H3 antibodies, anti-B7H4 antibodies or anti-B7H6 antibodies. In some embodiments, antibodies include B cell depleting antibodies. Typical B cell depleting antibodies include but are not limited to anti-CD20 monoclonal antibodies [e.g. Rituximab (Roche), Ibritumomab tiuxetan (Bayer Schering), Tositumomab (GlaxoSmithKline), AME-133v (Applied Molecular Evolution), Ocrelizumab (Roche), Ofatumumab (HuMax-CD20, Gemnab), TRU-015 (Trubion) and IMMU-106 (Immunomedics)], an anti-CD22 antibody [e.g. Epratuzumab, Leonard et al., Clinical Cancer Research (Z004) 10: 53Z7-5334], anti-CD79a antibodies, anti-CD27 antibodies, or anti-CD19 antibodies (e.g. U.S. Pat. No. 7,109,304), anti-BAFF-R antibodies (e.g. Belimumab, GlaxoSmithKline), anti-APRIL antibodies (e.g. anti-human APRIL antibody, ProSci inc.), and anti-IL-6 antibodies [e.g. previously described by De Benedetti et al., J Immunol (2001) 166: 4334-4340 and by Suzuki et al., Europ J of Immunol (1992) 22 (8) 1989-1993, fully incorporated herein by reference].

The immunotherapeutic treatment may consist of allografting, in particular, allograft with hematopoietic stem cell HSC. The immunotherapeutic treatment may also consist in an adoptive immunotherapy as described by Nicholas P. Restifo, Mark E. Dudley and Steven A. Rosenberg “Adoptive immunotherapy for cancer: harnessing the T cell response, Nature Reviews Immunology, Volume 12, April 2012). In adoptive immunotherapy, the subject's circulating lymphocytes, NK cells, are isolated amplified in vitro and readministered to the subject. The activated lymphocytes or NK cells are most preferably be the subject's own cells that were earlier isolated from a blood or tumor sample and activated (or “expanded”) in vitro.

In some embodiments, the subject once diagnosed as suffering from cancer is administered with a radiotherapeutic agent. The term “radiotherapeutic agent” as used herein, is intended to refer to any radiotherapeutic agent known to one of skill in the art to be effective to treat or ameliorate cancer, without limitation. For instance, the radiotherapeutic agent can be an agent such as those administered in brachytherapy or radionuclide therapy. Such methods can optionally further comprise the administration of one or more additional cancer therapies, such as, but not limited to, chemotherapies, and/or another radiotherapy.

In some embodiments, when it is determined that the subject will achieve a response dendrogenin A, the subject is then administered with said drug.

In some embodiments, the subject once diagnosed as suffering from cancer is administered with a nucleic acid encoding for hGSTA1. Typically, the nucleic acid encoding for hGSTA1 is delivered with a vector.

In its broadest sense, a “vector” is any vehicle capable of facilitating the transfer of the antisense oligonucleotide siRNA or ribozyme nucleic acid to the cells. Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the nucleic acid sequence of interest. Viral vectors are a preferred type of vector and include, but are not limited to nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in KRIEGLER (A Laboratory Manual,” W.H. Freeman C.O., New York, 1990) and in MURRY (“Methods in Molecular Biology,” vol. 7, Humana Press, Inc., Cliffton, N.J., 1991).

Preferred viruses for certain applications are the adeno-viruses and adeno-associated viruses, which are double-stranded DNA viruses that have already been approved for human use in gene therapy. The adeno-associated virus can be engineered to be replication deficient and is capable of infecting a wide range of cell types and species. It further has advantages such as, heat and lipid solvent stability; high transduction frequencies in cells of diverse lineages, including hematopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well known to those of skill in the art. See e.g., SANBROOK et al., “Molecular Cloning: A Laboratory Manual,” Second Edition, Cold Spring Harbor Laboratory Press, 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

Typically the active ingredient as described above is administered to the subject in a therapeutically effective amount.

By a “therapeutically effective amount” of the active ingredient as above described is meant a sufficient amount of the compound. It will be understood, however, that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidential with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

According to the invention, the active ingredient is administered to the subject in the form of a pharmaceutical composition. Typically, the active ingredient may be combined with pharmaceutically acceptable excipients, and optionally sustained-release matrices, such as biodegradable polymers, to form therapeutic compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The active ingredient can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the typical methods of preparation are vacuum-drying and freeze-drying techniques which yield a powder of the active ingredient plus any additional desired ingredient from a previously sterile-filtered solution thereof. The preparation of more, or highly concentrated solutions for direct injection is also contemplated, where the use of DMSO as solvent is envisioned to result in extremely rapid penetration, delivering high concentrations of the active agents to a small tumor area. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. Some variation in dosage will necessarily occur depending on the condition of the subject being treated. The person responsible for administration will, in any event, determine the appropriate dose for the individual subject.

The invention will be further illustrated by the following figures and examples. However, these examples and figures should not be interpreted in any way as limiting the scope of the present invention.

### FIGURES

FIG. 1: a) Dendrogenin A is the product of the enzymatic conjugation of 5,6α-epoxy-cholesterol (5,6α-EC) with histamine (HA). The reaction is an SN2 reaction resulting from the attack of the primary amine (a) of histamine (HA) on the C6 through the β side of the steroid backbone giving a 6β substitute product. b) The rat Glutathione S-transferase B (GST-B) catalyzed the stereoselective glutathionylation of 5,6α-EC to give 5α-dihydroxycholestan-6β-yl-S-glutathione (CDO-SG), a cholesterol metabolite with the precise stereochemistry of DDA at C6.

FIG. 2: Measurement of glutathionylation activity of hGSTA1. Glutathionylation of CDNB was done with recombinant hGSTA1 in the presence of (a) increasing concentrations of CDNB and a single GSH concentration or (b) increasing concentrations of GSH and a single CDNB concentration. c) The glutathionylation of CDNB was done in the presence of 1 and 10 μM 5,6α-EC or Histamine (HA). The glutathionylation of 5,6α-EC was done in the presence of (d) increasing hGSTA1 concentrations, (e) increasing GSH concentrations or (f) increasing 5,6α-EC concentrations. The reaction of CDO-S-G biosynthesis by hGSTA1 was carried out in the presence of (g) 1 and 10 mM CDNB or 1 and 10 μM HA, (h) or 1 μM 5,6β-EC or 10 μM CT, OCDO, 27-HC or 22(R)-HC.

FIG. 3: Measurement of the histaminylation of 5,6α-EC by hGSTA1. Histaminylation of 5,6α-EC was measured using recombinant hGSTA1 in the presence of (a) increasing concentrations of HA and a single 5,6α-EC concentration or (b) increasing concentrations of 5,6α-EC and a single HA concentration. (c) The product of this reaction was characterized by mass spectrometry and gave the expected mass of 514.4371 confirming the production of DDA by GSTA1. (d) The reaction of DDA biosynthesis by hGSTA1 was carried out in the presence of 10 mM CDNB with increasing concentrations of GSH ranging from 1 μM to 1 mM, (e) or 1 μM 5,6β-EC or 10 μM CT, OCDO, 27-HC or 22(R)-HC.

FIG. 4: Expression of GSTA1 in HEK293 induced DDAS activity. The expression of hGSTA1 by transfection of HEK293T cells with a plasmid encoding hGSTA1 (p-hGSTA1) was confirmed by Western blot (a) and immunocytochemistry (b). The functionality of hGSTA1 was validated by measurement of DCNB glutathionylation (c). (d) Transfected cells activated the biosynthesis of DDA and thus DDAS activity.

FIG. 5: Expression of DDAS and quantification of endogenous DDA in human breast tumors and normal tissues. Photomicrographs of immunohistochemical staining of DDAS expression in biopsies from patients. The stain is diaminobenzidine counterstained with hematoxylin. (a) DDAS is specifically expressed in (1) lactating duct terminal units (LTDU) and (3) in lactating ducts (LD), but not in tumor cells (1,3). DDAS is expressed in the cytoplasm of epithelial cells LD and LDTU (2, 4). (b) Quantification of DDAS expression in normal breast and in tumors after immunostaining was done as described in the “Methods” section (0 is no expression and 3 is high expression). DDA was quantified in tumors and adjacent normal tissues from 50 patients as described in the “Methods” section (c). Each symbol represents the mean concentration of DDA in each normal or tumor sample that was analyzed twice. The black line indicates the group sample mean±S.E., *P<0.001. (d) Paired comparison of the level of DDA between normal tissue and the corresponding tumor (p=0.024, n=50, paired Wilcoxon signed rank test).

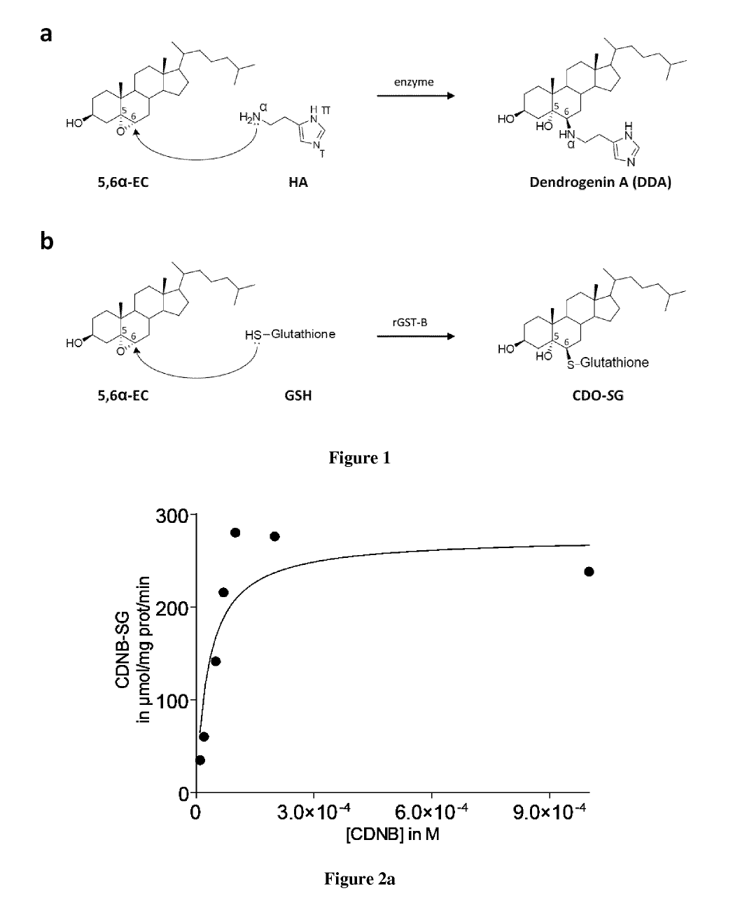

### EXAMPLE

Material & Methods

Chemicals.

[¹⁴C]cholesterol (58 mCi/mmol) and [³H]cholesterol (53 Ci/mmol) were purchased from Perkin Elmer. Sterols, oxysterols and drugs were from Steraloids or Sigma-Aldrich. DDA and 5,6α-EC and 5,6β-EC were synthesized in our laboratory according to published procedures^{10,27}. 3β,5α-dihydroxycholestan-6β-yl-S-glutathione (CDO-SG) was synthesized as previously described¹⁴ and was 99% pure by TLC. Solvents were from Sigma, Fischer, Scharlau or VWR. TLC plates were from Macherey Nagel. C18 Sep-Pack cartridges were from Waters.

Measurement of GSTA1 Activity.

Glutathione S-transferase activity was measured according to the method of Habig et al²⁸. Briefly, 2 μg of hGSTA1 (NBP1-30307, Novus company) were incubated in the presence of 1 mM glutathione in 0.1 M potassium phosphate buffer (pH 7.0) and 100 μM 1-chloro-2,4-dinitrobenzene (CDNB, Sigma) in ethanol (2.85% vol/vol). The mixture was incubated for 30 min at 30° C. and the reaction was stopped with 200 μl MeOH. Measurements were carried out spectrophotometrically at 345 nm (Aₘ=8.5 mM⁻¹·cm⁻¹). The negative control corresponds to the same test performed in absence of hGSTA1.

Measurement of CDO-SG and DDA Biosynthesis.

The final assay volume of 100 μl with 0.1 M potassium phosphate buffer (pH 7.5), 2 μg hGSTA1 (1 μg/μ1), was incubated with increasing concentrations of [³H]5,6α-EC (50 nM to 500 nM), GSH (1 μM to 1 mM) or HA (0.1 to 100 μM). The mixture was incubated for 30 minutes at 30° C., and the reaction was stopped by addition of 200 μl MeOH. After centrifugation 10 min at 18,000 g (4° C.), the supernatant was evaporated to dryness using a speed vac concentrator (Savant SP01010, Thermo scientific) and the residue was resuspended in 20 μl MeOH. Samples were spotted on HPTLC NanoSilgur 10×10 cm plates (Macherey Nagel, ref 811042) and developed using using ethyl acetate/butanol/acetic acid/water (6:2:3:2) for CDO-SG and CH₂Cl/MeOH/NH₄OH 84:15:1 for DDA. Standards were co-spotted with samples. Plates were revealed by sulfuric acid impregnation and heating. The radioactivity was quantified by liquid scintillation counting of the ethanol extract of the silica scraped at the retention factor (Rf) corresponding to authentic 5,6α-EC, CDO-SG or DDA standards.

Cell Culture.

HEK293T cells were from the ATCC. HEK293T cells were grown in RPMI 1640 medium supplemented with 1.2 mM glutamine, 5% fetal bovine serum, penicillin, and streptomycin (50 units/mL) in a humidified atmosphere with 5% CO₂ at 37° C. Expression of hGSTA1 in HEK293T. Cells were transfected using the Neon™ transfection system following the instructions of the Invitrogen Company (program 13, 5.10⁶ cells). Cells were transfected with 4 μg of a mock plasmid (pCMV6-AC, PS100020, Origene Company) or a plasmid encoding hGSTA1 (pCMV6-hGSTA1, SC321900, Origene Company). Proteins were separated on 4-12% SDS-PAGE gels (NuPAGE Novex Invitrogen 4-12% Bis-Tris protein gels, NP0335BOX), electro-transferred onto PVDF membranes and incubated overnight at 4° C. with rabbit anti-GSTA1 (PA5-29811, Thermo Scientific) or anti-Actin (C4, MAB1501, Millipore). Visualization was carried out using an ECL plus kit (Pierce), and revealed with the ChemiDoc™ MP Imaging System (Biorad). Immunocytochemistry was carried as previously described¹⁰ using an anti-hGSTA1 (PA5-29811, Thermo Scientific, dilution1/500) and revealed with an anti-HRP secondary antibody (Abeam, Anti-Rabbit IgG, 1/500) after Giemsa staining.

Measure of DDA Biosynthesis in Cells Transfected with hGSTA1.

Transfected cells were seeded in 6-well plates at 100 000 cells per well in triplicate. After 24 h, the cells were incubated with [14C]-5,6αEC (0,072 μCi) in EtOH 0.1%. After 48 h, the cells were incubated with HA at 100 μM for 96 h. Cells were then scraped and pelleted by centrifugation for 5 min at 1,400 rpm, and then extracted, developed and analyzed as described with recombinant hGSTA1.

MS Experiments.

Mass spectrometry experiments were performed on an Exactive mass spectrometer (Thermo Scientific) equipped with a HESi-II probe electrospray ionization source operating in positive mode. Samples were introduced using a syringe pump and a 500 μL syringe (Thermo scientific) at a flow rate of 10 μL/min. The Exactive MS was tuned using a 100 fmol/μL Dendrogenin A solution to optimize parameters. Optimized parameters were as follows: spray voltage, 3.8 kV; heated capillary temperature, 150° C.; capillary voltage, 82.50 V; tube lens voltage, 185 V; skimmer voltage, 34 V, sheath gas (nitrogen) flow rate, 20 (arbitrary units); auxiliary gas flow rate, 10 (arbitrary units). The instrument was set to a maximum injection time of 50 ms with 2 microscans per spectrum. The data were acquired via the Thermo Exactive Tune software.

Preparation of Tissues.

All human samples were collected with the approval of institutional review board of the Purpan University Hospital and of the Claudius Regaud Institute. Normal and tumor human tissues obtained by surgical resection were stored frozen at −80° C. and thawed in ice before use. Patients' clinical characteristics and tumor pathological features were obtained from the medical reports and followed the standard procedures in our institution. Tissues were homogenized at 4° C. in buffer A (50 mM Tris, 0.5% butylated hydroxytoluene (5 mg ml⁻¹), 150 mM KCl, pH 7.4) (5 vol g⁻¹ of tissue) with a Polytron B homogenizer and centrifuged 5 min at 2,500 r.p.m. Homogenates and sera were diluted with buffer A to obtain samples at 10 mg protein per ml. Quantification of DDA from tumors and normal matching tissues was done exactly as previously described¹⁰.

Immunohistochemistry.

Human tissues were analysed for hGSTA1 expression. Immunohistochemical staining was done on paraffin-embedded tissue sections, using a specific anti-human hGSTA1 antibody (PA5-29811, Thermo Scientific). Immunostaining of paraffin sections was preceded by an antigen retrieval technique by heating in 10 mM citrate buffer, pH 6, with a microwave oven twice for 10 min each time. After incubation with the antibody for 1 h at room temperature, sections were incubated with biotin-conjugated polyclonal anti-rabbit immunoglobulin antibody followed by the streptavidin-biotin-peroxidase complex (Vectastain ABC kit, Vector Laboratories, CA) and were then counterstained with hematoxylin. Negative controls were incubated in buffered solution without primary antibody. Immunohistochemistry was performed on 3-μm-thick representative whole tissue sections from 50 cases, which were mounted on polylysine-coated slides. hGSTA1 antibody (PA5-29811, ThermoScientific) was used at 1:100. Heat-induced antigen retrieval was employed: 30 min in buffer pH 6.0 in a pre-treatment module (Labvision, Fremont, Calif., USA) for hGSTA1 antibody. Sections were blocked with 1.5% H₂O₂ in methanol for 10 min and incubated with hGSTA1 antibodies for 60 min at room temperature. Detection was achieved with the Vector avidin-biotin complex (ABC) system (Vector Laboratories, Burlingame, Calif., USA) according to the manufacturer's recommendations, using 3,3′-diaminobenzidine (Dako, Glostrup, Denmark) as a chromogenic substrate. Slides were lightly counterstained with haematoxylin. Positive controls (normal breast section) and negative controls (omission of the primary antibody and substitution of the primary antibody by IgG-matched control) were included in each experiment. hGSTA1 immunostains obtained with the two antibodies were analyzed independently by five of the authors (MV, SSP, MP, FD and MLT) using the Allred scoring system that combines the staining intensity and the percentage of stained cells (intensity score 0-3+% score 0-5)²⁹. For each case, the score was assessed separately for the cytoplasmic, nuclear and membrane reactivity. An Allred score of >2 was considered as positive. Immunohistochemical analysis with the hGSTA1 clone was carried out with the observers blinded to the results of the analysis of the HEK23T-hGSTA1 clone. For a comparison with fibromatosis, we considered the expression patterns and levels of β-catenin in the stromal cells of phyllodes tumors and in the most abundant component of metaplastic carcinomas. Data analysis was performed with the results obtained with each antibody and also with combined results (ie, for nuclear/cytoplasmic staining, positivity was defined as positive for at least one antibody; for membrane staining, the highest Allred score was taken into account).

Statistical Analysis.

Values are the mean±S.E. of three independent experiments each carried out in duplicate. Statistical analysis was carried out using a Student's t-test for unpaired variables. * and ** in the figures refer to statistical probabilities (P) of <0.001 and <0.0001, respectively, compared with control cells that received solvent vehicle alone. Tissues from patients were analyzed for significance and pairing with Wilcoxon signed rank tests. In the figures, *, ** and *** refer to P<0.05, P<0.01 and P<0.001, respectively, compared with controls (vehicle) unless otherwise specified. Prism software was used for all the analyses.

Results

hGSTA1 Catalyzed DDA Biosynthesis

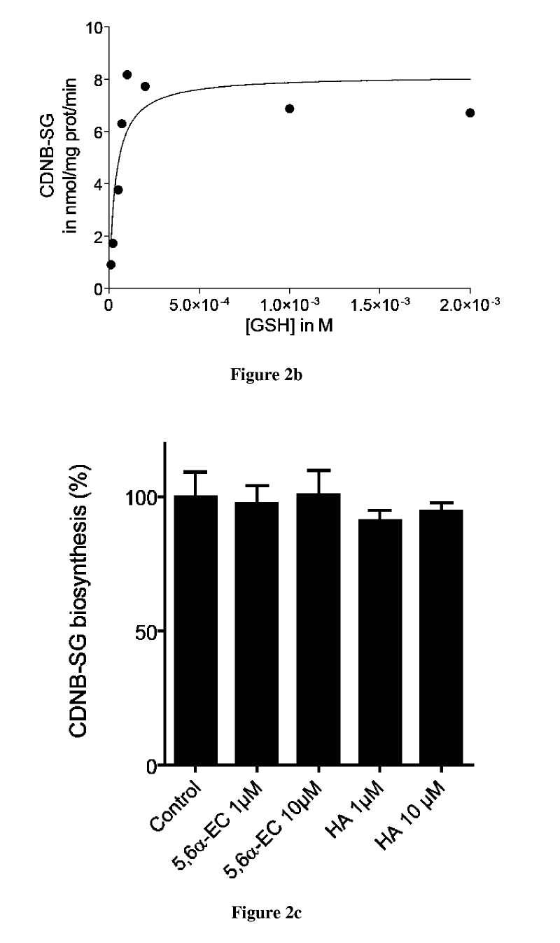

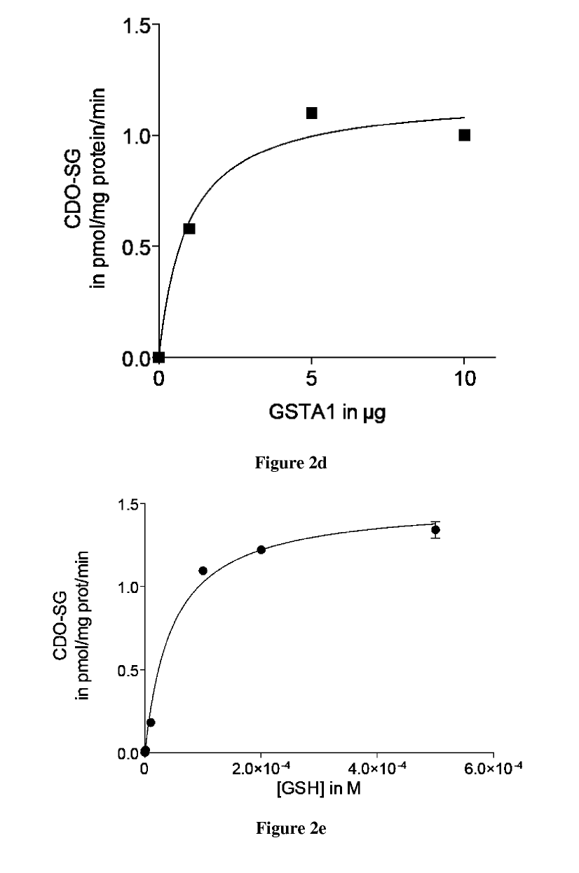

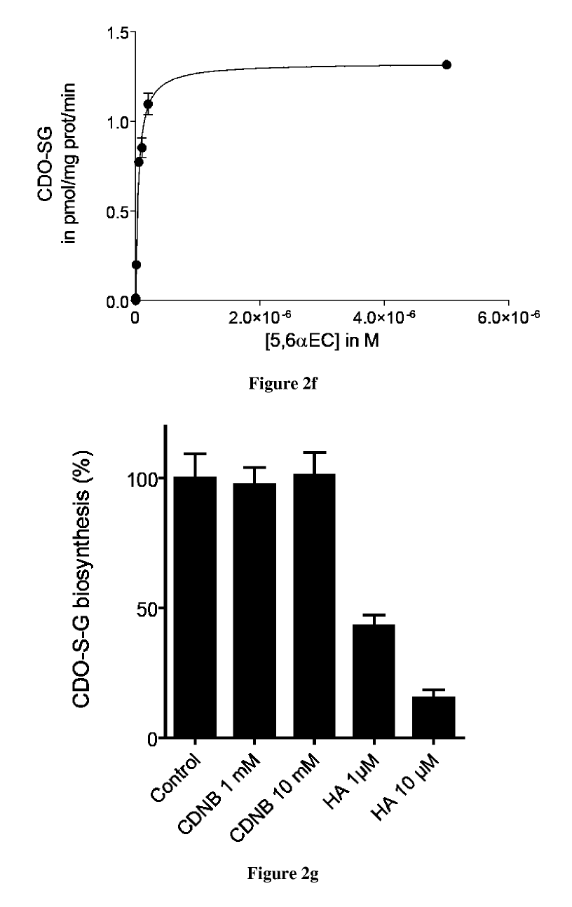

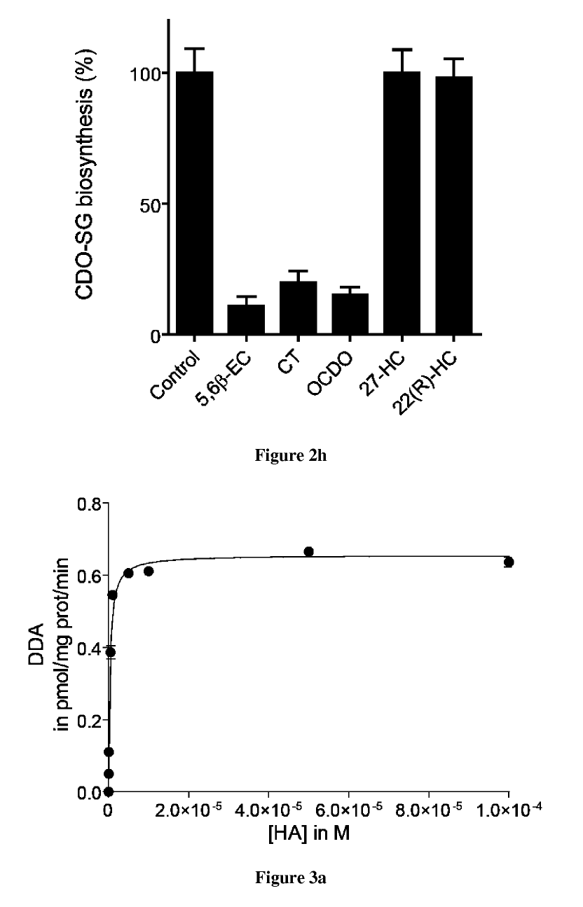

Commercially available hGSTA1 was found to be functionally active and catalyzed the conjugation of the xenobiotic 1-chloro-2,4-dinitrobenzene (CDNB) with GSH to give S-(2,4-dinitrophenyl)-glutathione (CDNB-SG) with Kₘ^{CDNB}=32.2 μM, Vₘ^{CDNB}=274 μmol·min⁻¹·mg prot for CDNB and Kₘ^{GSH}=35.1 μM, Vₘ^{GSH}=8.12 μmol·min⁻¹·mg prot for GSH (FIG. 2a-b and table 1). 5,6-EC and HA were not inhibitors of CDNB-SG biosynthesis (FIG. 2c). We therefore tested whether hGSTA1 could catalyze the formation of CDO-SG from [¹⁴C]-5,6-EC and GSH as reported for rat GST B^{14,15.} We showed that hGSTA1 catalyzed the biosynthesis of CDO-SG with a Kₘ^{5,6EC}=0.44 μM and Kₘ^{GSH}=47 μM, while no reaction occurred in the absence of the enzyme establishing its obligatory requirement for CDO-SG biosynthesis (FIG. 2d-f, and table 1). We found that hGSTA1 had a 73-fold higher affinity for 5,6-EC than for CDNB while the Kₘ^{GSH} was in the same range for both catalytic activities. The maximum velocities were Vₘ^{5,6-EC}=1.33 pmol·min⁻¹·mg prot Vₘ^{GSH}=1.50 pmol·min⁻¹·mg prot (Table 1) showing a 24.10⁶ fold weaker capacity to produce CDO-SG than to produce CDNB-SG. CDNB did not inhibit CDO-SG biosynthesis while HA was a potent inhibitor of the reaction (FIG. 2g) suggesting that HA was a possible substrate of hGSTA1. We next found that ring B oxysterols such as 5,6β-EC, CT and OCDO were potent inhibitors of CDO-SG biosynthesis while side chain oxysterols were not inhibitors up to 10 μM (FIG. 2h).

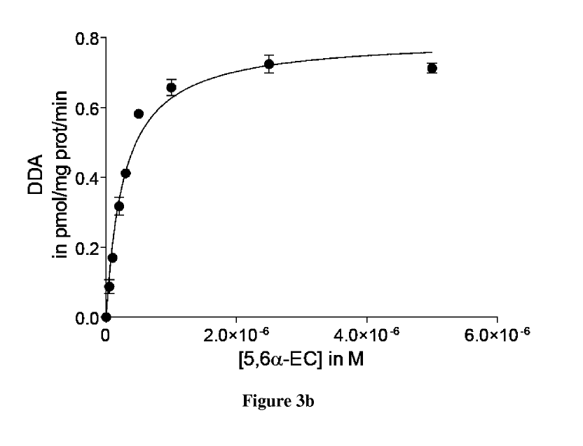

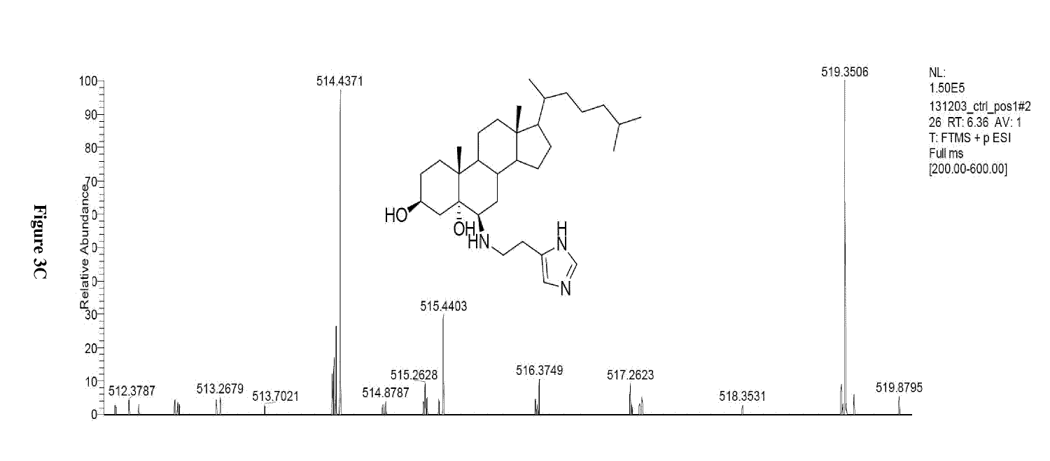

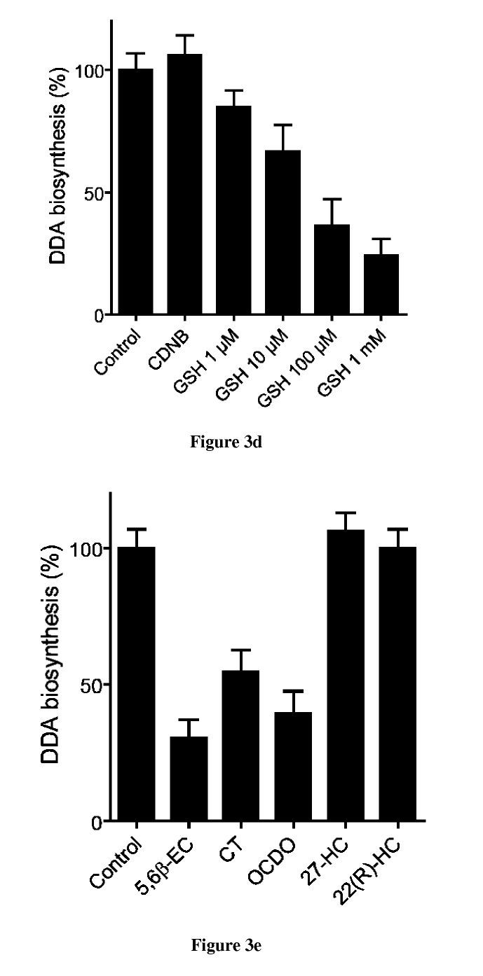

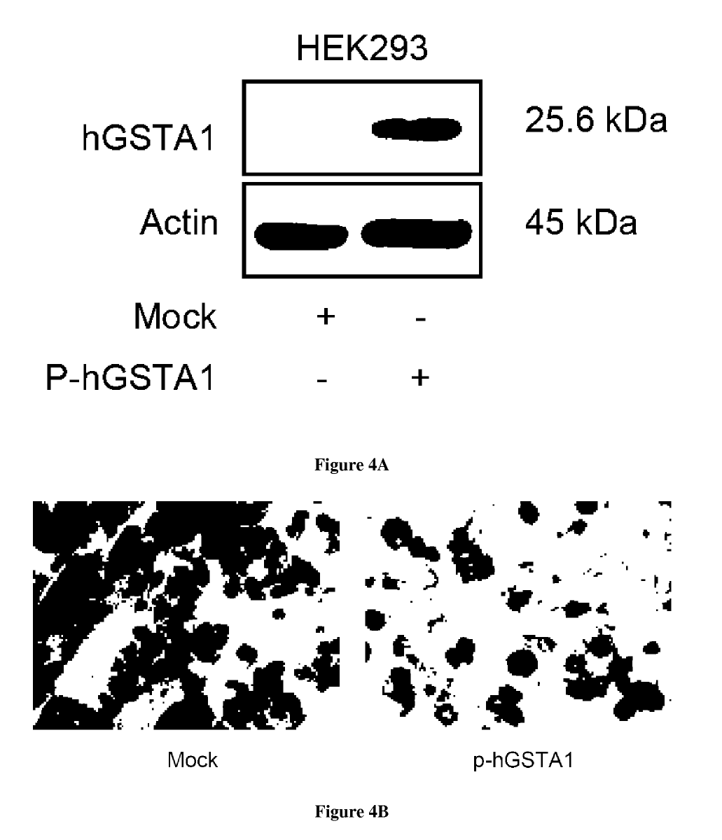

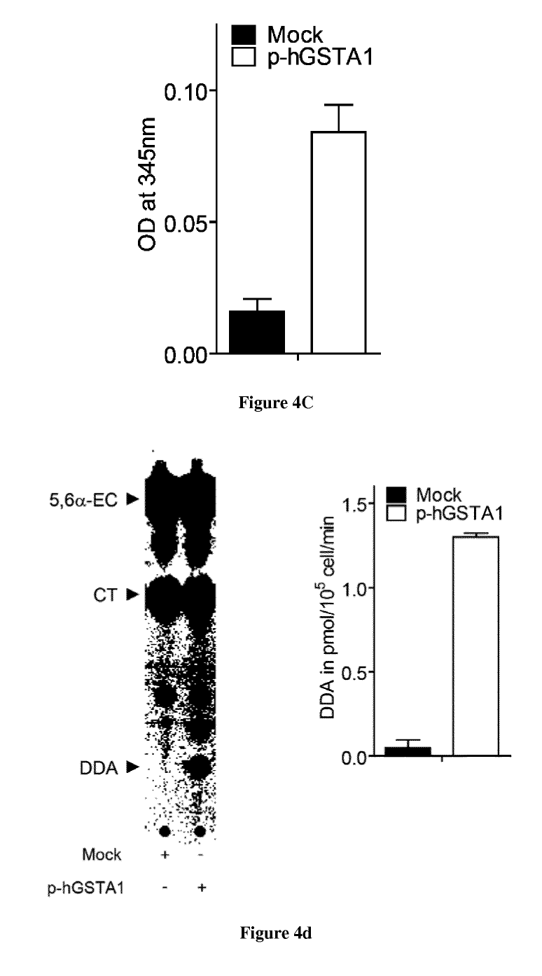

We next showed that hGSTA1 catalyzed the biosynthesis of DDA from [¹⁴C]-5,6-EC and HA with a Kₘ^{5,6EC}=0.28 μM and Kₘ^{HA}=0.35 μM and maximum velocities: Vₘ^{5,6EC}=0.81 pmol·min⁻¹·mg prot and Vₘ^{HA}=0.66 pmol·min⁻¹·mg prot (FIG. 3a-b, Table 1). No reaction occurred in the absence of enzyme as opposed to what was found for xenobiotic conjugation that occurred spontaneously. While hGSTA1 is known to facilitate or to accelerate the conjugation reaction of the CDNB with GSH, we showed here that DDAS is absolutely required for DDA biosynthesis. The production of DDA by hGSTA1 was confirmed by mass spectrometry giving a MS1 parent peak with a mass of 514 that gave a fragment of 496 in MS2 (FIG. 3c), which is characteristic of DDA¹⁰. hGSTA1 had a comparable affinity for 5,6α-EC and HA, in the range of their reported endogenous concentrations ([5,6-EC]=0.5 μM⁶, [HA]=12 μM¹⁷). Next we showed that GSH inhibited the biosynthesis of DDA dose-dependently giving an IC₅₀ of 52 μM while CDNB did not inhibit this reaction and that ring B oxysterols but not side chain oxysterols were inhibitors of DDAS (FIG. 3d). 5,6β-EC was found to be the most potent inhibitor of the oxysterol series we tested (FIG. 3e). To determine whether hGSTA1 catalyzed DDAS in cells, a plasmid encoding hGSTA1 was expressed in HEK293 cells. The expression of the enzyme was confirmed by Western blotting (FIG. 4a) and immunocytochemistry showed that the enzyme was mainly cytoplasmic and nuclear (FIG. 4c). We found that hGSTA1-transfected cells glutathionylated CDNB (FIG. 4A) and, importantly, showed DDAS activity, establishing that hGSTA1 catalyzed DDA biosynthesis in intact cells (FIG. 4d). Altogether, these data established that hGSTA1 catalyzed DDA biosynthesis.

DDAS is Selectively Expressed in the Epithelial Cells of Lactating Duct and Lobular Terminal Units.

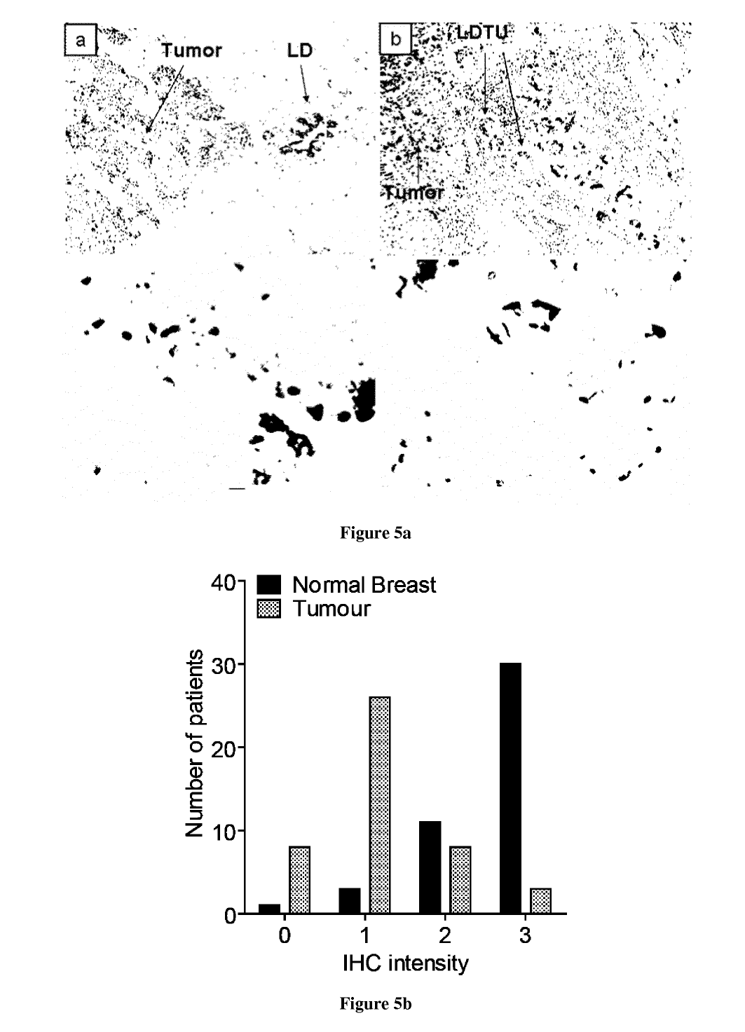

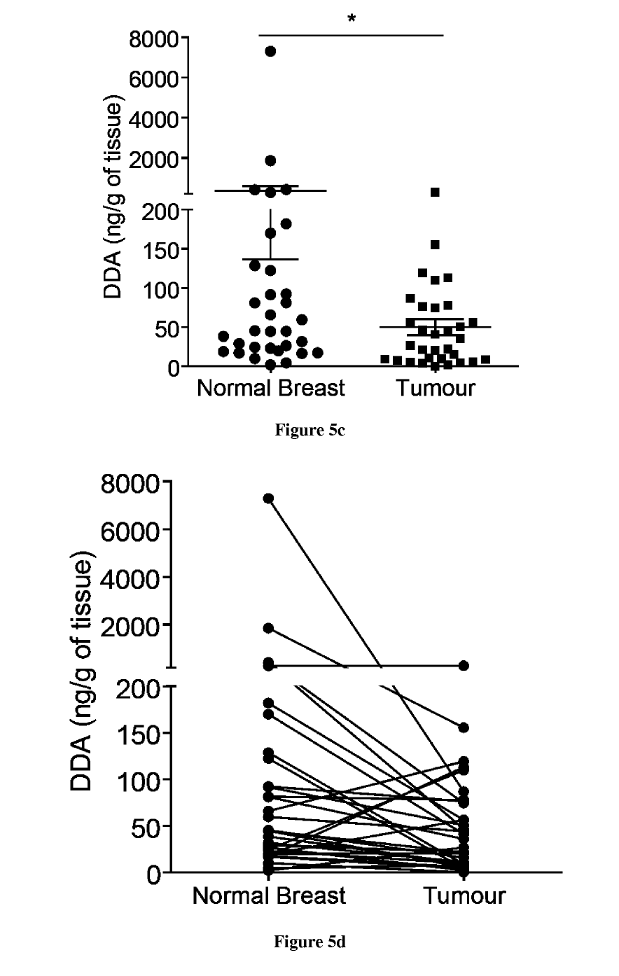

We previously found DDA in whole extracts of normal breast¹⁰ but the nature of the cells producing DDA remained to be established. We then analysed DDAS immuno-histochemically in normal breast tissues from patients. Fifty normal breast tissue samples were found to be 97.9% positive for DDAS, which was selectively expressed in the epithelial cells of lactating ducts and lobular terminal units from all samples. DDAS was not found in adipocytes, endothelial cells from vessels, fibroblasts or myo-epithelial cells. The cellular localization of DDAS in epithelial cells was mainly cytoplasmic (FIG. 5a) and in 4% of the samples was also found in the nuclei. These data established that DDAS is selectively expressed in the epithelial cells of lactating ducts and lobular terminal units thus identifying the source of DDA production in the breast.

DDAS is not Expressed in Breast Cancer (BC) Cells and Tumors.

We previously showed that BC cell lines did not produce DDA¹⁰ and consistently we found that these cells do not express DDAS (supplementary Table 1). We next analyzed 50 tumors from patients with BC by IHC and showed that DDAS expression was considerably decreased in the tumors of patients (FIG. 5b) compared to normal matched tissues. Analysis of the DDA levels in tumors showed a mean 8-fold decrease in DDA from 425±288 to 49.9±10.7 ng/mg tissue (FIG. 3c), importantly, the measured decreased in DDA between the normal tissue and the matched tumor tissues was paired (Wilcoxon test, p<0.005) (FIG. 3c). These data established that DDAS was not expressed in BC cells and considerably decreased in BC tumors and this decrease paralleled the decrease in DDA we measured.

Discussion

DDA is a steroidal alkaloid recently discovered in mammals as the product of an enzyme-catalyzed conjugation of 5,6α-EC with the α-amino group of HA¹⁰. The discovery of DDA was important because DDA posesses TS properties, which has never been reported for a cholesterol metabolite. Moreover, DDA is the first mammalian steroidal alkaloid found to date′° defining a new class of steroids in mammals and a new class of LXR endogenous ligands with sub-μM affinity^{8,9}.

DDA was not found in BC cell lines and found in lower amounts in BC biopsies establishing a deregulation of DDA metabolism in BC. The identification of the DDA synthase (DDAS) was thus a crucial challenge to understand this deregulation.

We hypothesized that hGSTA1 was a possible candidate for DDAS activity, because its rat orthologue GST-B was reported to catalyze a similar reaction using 5,6α-EC and GSH as a nucleophile. Two different groups showed that GST-B catalyzed the stereoselective biosynhesis 5α-dihydroxycholestan-6β-yl-S-glutathione (CDO-SG), which displays the same C5αOH-C6β-substituted stereochemistry than DDA^{14,15} We report in the present study its human ortholog (hGSTA1) catalyzed also CDO-SG biosynthesis, establishing that human enzyme catalyzed the same reaction than the rat enzyme and used 5,6α-EC and GSH as endogenous substrates. The Kₘ obtained for 5,6α-EC transformation was 0.44 μM which is consistent with an endogenous concentration of 0.5 μM in 5,6α-EC measured in mammalian tissues⁶. Replacing of GSH by HA, we found that hGSTA1 catalyzed the biosynthesis of DDA from 5,6α-EC with a similar Km for 5,6α-EC and a 135 fold weaker Kₘ for HA than for GSH, establishing a preference of hGSTA1 for HA over GSH as substrate. Again, the Kₘ obtained for HA transformation was 0.35 μM which is consistent with the endogenous concentration found for HA in human breast¹⁷. We found that the xenosubstrate of hGSTA1 CDNB did not inhibit CDO-SG and DDA biosynthesis, strongly suggesting that the binding sites for CDNB and 5,6α-EC were different. The physiological function previously attributed to hGSTA1 was xenobiotic detoxification, steroidogenesis¹⁸ and elimination of 5,6α-EC by conjugation with GSH¹⁴. GSTA1 has a catalytic efficiency 60 fold higher to transform 5,6α-EC compared to CDNB, and 60 fold higher to transform HA compared to GSH. We next established that the expression of GSTA1 in cells induced DDAS activity. Altogether these data showed that GSTA1 catalyzed DDAS activity, which maybe the primary function of GSTA1.

The fact that 5,6β-EC, the diasteroisomer of 5.6α-EC, was a potent inhibitor of DDAS is interesting, because it can potentially impact on DDA biosynthesis. 5,6β-EC is the major product of ROS activated lipoperoxidation^{6,19} suggesting that inflammation may be detrimental to the production of the tumor suppressor DDA and thus can contribute to carcinogenicity. In the late seventies, Watabe's group reported the existence of a cholesterol 5,6α epoxidase²⁰, which supports the existence of a metabolic pathways contributing to DDA biosynthesis from cholesterol. They showed that a yet unidentified cytochrome P450 catalyzed the stereoselective biosynthesis 5.6α-EC²⁰. Further studies are now required to identify this cytochrome p450.

Normal breast is a heterogeneous tissue containing different types of cells. HIC studies revealed that DDAS is selectively expressed in the cytoplasm of epithelial cells from lactiferous ducts and lobules giving the cellular origin of DDA production in the breast. The absence of DDAS in BC tumors explains the decrease in the DDA content found in tumors. The absence of DDA in BC tissue due to a lack of DDAS expression is consistent with the increased amount of HA^{17,21} and 5,6-EC²² measured in ductal breast cancer and breast epithelial hyperplasic tissues respectively. Statins are broadly used anti-cholesterol drugs prescribed for the prevention of cardiovascular diseases²³, but their benefit in the chemoprevention of breast cancer is still a matter of debate²⁴. Since long term statin use was recently reported to increase the risk of both invasive ductal carcinoma and invasive lobular carcinoma²⁵, in which we found that DDAS expression was reduced, it raises the important question of the impact of statins on the DDA content in the breast and deserves further investigations.

We found that DDAS expression reflected DDA levels in breast cancer tissues. The accuracy of the quantification of DDA levels by LC/MS in BC tumors is limited by possible contamination with normal tissues; in addition this method is laborious and expensive. The identification of DDAS enabled the measurement of its expression in BC by immunohistochemistry. This approach will be cheaper and more precise than the quantification of DDA and useful for histopathology platforms.

DDA has been detected in different mammalian organs such as lung, brain and spleen but found to be absent from their corresponding tumor cell lines¹⁰. Further studies will be required to determine if this correlates with the loss of DDAS activity.

DDA was active on tumor cell lines of different origins that do not contain measurable amounts of DDA⁸⁻¹¹ and that do not or only weakly express GSTA1²⁶. Thus the absence of GSTA1 and the expression of LXRβ, the molecular target of DDA, may be predictive of a response to DDA.

In summary, we report in the present study the first identification and molecular identification of DDAS: the enzyme that catalyzes the biosynthesis of the tumor suppressor dendrogenin A.

TABLE 1
Enzymatic parameters of hGSTA1 for exogenous and endogenous substrates.

| Substrates | | Product | Kₘ | | Vₘ | |
|---|---|---|---|---|---|---|
| | | | Kₘ^{CDNB} | Kₘ^{GSH} | Vₘ^{CDNB} | Vₘ^{GSH} |
| | | | (μM) | (μM) | (μmol · min⁻¹ · mg⁻¹) | (μmol · min⁻¹ · mg⁻¹) |
| CDNB | GSH | CDNB-S-G | 32.2 | 35.1 | 274 | 8.12 |
| | | | Kₘ^{5,6-EC} | Kₘ^{GSH} | Vₘ^{5,6-EC} | Vₘ^{GSH} |
| | | | (μM) | (μM) | (pmol · min⁻¹ · mg.pr) | (pmol · min⁻¹ · mg · pr) |
| 5,6α-EC | GSH | CDO-SG | 0.44 | 47 | 1.33 | 1.50 |
| 5,6β-EC | GSH | — | — | — | — | — |
| | | | Kₘ^{5,6-EC} | Kₘ^{His} | Vₘ^{5,6-EC} | Vₘ^{His} |
| | | | (μM) | (μM) | (pmol · min⁻¹ · mg.pr) | (pmol · min⁻¹ · mg · pr) |
| 5,6α-EC | His | DDA | 0.28 | 0.35 | 0.81 | 0.66 |
| 5,6β-EC | His | — | — | — | — | — |

CDNB: 1,chloro-2,4-dinitrobenzene;
GSH: glutathione;
CDNB-S-G: S-(2,4-dinitrophenyl)-glutathione
5,6α-EC: 5,6α-epoxy-cholesterol;
5,6βEC: 5,6β-epoxy-cholesterol;
His: histamine;
CDO-SG: 5α-dihydroxycholestan-6β-yl-S-glutathione;
dendrogenin A (DDA): 5α-hydroxy-6β-[2-(1H-imidazol-4-yl) ethylamin] cholestan-3β-ol;
5α-hydroxy-6β-[S-glutathione] cholestan-3β-ol,3β,

SUPPLEMENTARY TABLE 1
Quantification of GSTA1 in normal and tumor cells.

| | | GSTA1 | |
|---|---|---|---|
| Cell line | Cell type | expression | activity |
| MCF-7 | Breast carcinoma, human | — | N.M. |
| TS/A | Breast carcinoma, mouse | — | N.M. |
| SKBr3 | Breast carcinoma, human | — | N.M. |
| ZR75-1 | Breast carcinoma, human | — | N.M. |
| BT474 | Breast carcinoma, human | — | N.M. |
| HCC 1937 | Breast carcinoma, human | — | N.M. |
| MDA-MB-231 | Breast carcinoma, human | — | N.M. |

*Mean ± S.E.M. values (see Methods)
N.M., not measurable.

### REFERENCES

Throughout this application, various references describe the state of the art to which this invention pertains. The disclosures of these references are hereby incorporated by reference into the present disclosure.
1. Wu, Q. et al. 27-Hydroxycholesterol promotes cell-autonomous, ER-positive breast cancer growth. Cell Rep 5, 637-45 (2013).
2. Nelson, E. R. et al. 27-Hydroxycholesterol links hypercholesterolemia and breast cancer pathophysiology. Science 342, 1094-8 (2013).
3. Yue, S. et al. Cholesteryl ester accumulation induced by PTEN loss and PI3K/AKT activation underlies human prostate cancer aggressiveness. Cell Metab 19, 393-406 (2014).
4. de Medina, P. et al. Auraptene is an inhibitor of cholesterol esterification and a modulator of estrogen receptors. Mol Pharmacol 78, 827-36 (2010).
5. Paillasse, M. R. et al. Signaling through cholesterol esterification: a new pathway for the cholecystokinin 2 receptor involved in cell growth and invasion. J Lipid Res 50, 2203-11 (2009).
6. Poirot, M. & Silvente-Poirot, S. Cholesterol-5,6-epoxides: chemistry, biochemistry, metabolic fate and cancer. Biochimie 95, 622-31 (2013).
7. Silvente-Poirot, S. & Poirot, M. Cholesterol epoxide hydrolase and cancer. Curr Opin Pharmacol 12, 696-703 (2012).
8. Segala, G. et al. Dendrogenin A links Liver X Receptor to autophagic cell death in melanoma. submitted (2014).
9. Segala, G. et al. Abstract 1662: Dendrogenin A is a newly identified mammalian steroidal alkaloid that induced autophagic cell death in melanoma cells through an LXRbeta-, Nur77- and Nor1-dependent way. Cancer Research 73, 1662 (2013).
10. de Medina, P. et al. Dendrogenin A arises from cholesterol and histamine metabolism and shows cell differentiation and anti-tumour properties. Nat Commun 4, 1840 (2013).
11. de Medina, P., Paillasse, M. R., Payre, B., Silvente-Poirot, S. & Poirot, M. Synthesis of new alkylaminooxysterols with potent cell differentiating activities: identification of leads for the treatment of cancer and neurodegenerative diseases. J Med Chem 52, 7765-77 (2009).
12. Khalifa, S. A. et al. The novel steroidal alkaloids dendrogenin A and B promote proliferation of adult neural stem cells. Biochem Biophys Res Commun 446, 681-6 (2014).
13. Paillasse, M. R. et al. Surprising unreactivity of cholesterol-5,6-epoxides towards nucleophiles. J Lipid Res 53, 718-25 (2012).
14. Watabe, T., Sawahata, T. & Horie, J. Evidence for the formation of a steroid S-glutathione conjugate from an epoxysteroid precursor. Biochem Biophys Res Commun 87, 469-75 (1979).
15. Meyer, D. J. & Ketterer, B. 5 alpha,6 alpha-Epoxy-cholestan-3 beta-ol (cholesterol alpha-oxide): A specific substrate for rat liver glutathione transferase B. FEBS Lett 150, 499-502 (1982).
16. Mannervik, B., Board, P. G., Hayes, J. D., Listowsky, I. & Pearson, W. R. Nomenclature for mammalian soluble glutathione transferases. Methods Enzymol 401, 1-8 (2005).
17. Reynolds, J. L. et al. Histamine in human breast cancer. Br J Surg 85, 538-41 (1998).
18. Mannervik, B. Five decades with glutathione and the GSTome. J Biol Chem 287, 6072-83 (2012).
19. Segala, G. et al. 5,6-Epoxy-cholesterols contribute to the anticancer pharmacology of tamoxifen in breast cancer cells. Biochem Pharmacol 86, 175-89 (2013).
20. Watabe, T. & Sawahata, T. Biotransformation of cholesterol to cholestane-3beta,5alpha,6beta-triol via cholesterol alpha-epoxide (5 alpha,6alpha-epoxycholestan-3beta-ol) in bovine adrenal cortex. J Biol Chem 254, 3854-60 (1979).
21. von Mach-Szczypinski, J., Stanosz, S., Sieja, K. & Stanosz, M. Metabolism of histamine in tissues of primary ductal breast cancer. Metabolism 58, 867-70 (2009).
22. Wrensch, M. R. et al. Breast fluid cholesterol and cholesterol beta-epoxide concentrations in women with benign breast disease. Cancer Res 49, 2168-74 (1989).
23. LaRosa, J. C., He, J. & Vupputuri, S. Effect of statins on risk of coronary disease—A meta-analysis of randomized controlled trials. Jama-Journal of the American Medical Association 282, 2340-2346 (1999).
24. Silvente-Poirot, S. & Poirot, M. Cancer. Cholesterol and cancer, in the balance. Science 343, 1445-6 (2014).
25. McDougall, J. A. et al. Long-term statin use and risk of ductal and lobular breast cancer among women 55 to 74 years of age. Cancer Epidemiol Biomarkers Prev 22, 1529-37 (2013).
26. Tew, K. D. et al. Glutathione-associated enzymes in the human cell lines of the National Cancer Institute Drug Screening Program. Mol Pharmacol 50, 149-59 (1996).
27. Voisin, M., Silvente-Poirot, S. & Poirot, M. One step synthesis of 6-oxo-cholestan-3beta,5alpha-diol. Biochem Biophys Res Commun 446, 782-5 (2014).
28. Habig, W. H. et al. The identity of glutathione S-transferase B with ligandin, a major binding protein of liver. Proc Natl Acad Sci USA 71, 3879-82 (1974).
29. Harvey, J. M., Clark, G. M., Osborne, C. K. & Allred, D. C. Estrogen receptor status by immunohistochemistry is superior to the ligand-binding assay for predicting response to adjuvant endocrine therapy in breast cancer. J Clin Oncol 17, 1474-81 (1999).

1. A method of diagnosing cancer in a subject comprising the steps of
i) determining an expression level of hGSTA1 in a tumor sample obtained from the subject,
ii) comparing the expression level determined at step i) with its predetermined reference value and
iii) concluding that the subject suffers from a cancer when the expression level of hGSTA1 is lower than its predetermined reference value.

2. A method for determining the survival time of subject suffering from a cancer comprising the steps of
i) determining an expression level of hGSTA1 in a tumor sample obtained from the subject,
ii) comparing the expression level determined at step i) with its predetermined reference value and
iii) concluding that the subject will have a long survival time when the expression level of hGSTA1 is higher than its predetermined reference value or concluding that the subject will have a short survival time when the expression level of hGSTA1 is lower than its predetermined reference value.

3. A method for determining whether a subject suffering from a cancer will achieve a response with dendrogenin A of comprising
i) determining an expression level of hGSTA1 in a tumor sample obtained from the subject,
ii) comparing the expression level determined at step i) with its predetermined reference value and
iii) concluding that the subject will achieve a response with dendrogenin A when the expression level of hGSTA1 is lower than its predetermined reference value.

4. The method of claim 1, wherein the cancer is selected from the group consisting of bile duct cancer, bladder cancer, bone cancer, brain and central nervous system cancer, breast cancer, Castleman disease, cervical cancer, colorectal cancer, endometrial cancer, esophagus cancer, gallbladder cancer, gastrointestinal carcinoid tumors, Hodgkin's disease, non-Hodgkin's lymphoma, Kaposi's sarcoma, kidney cancer, laryngeal and hypopharyngeal cancer, liver cancer, lung cancer, mesothelioma, plasmacytoma, nasal cavity and paranasal sinus cancer, nasopharyngeal cancer, neuroblastoma, oral cavity and oropharyngeal cancer, ovarian cancer, pancreatic cancer, penile cancer, pituitary cancer, prostate cancer, retinoblastoma, rhabdomyosarcoma, salivary gland cancer, skin cancer, stomach cancer, testicular cancer, thymus cancer, thyroid cancer, vaginal cancer, vulvar cancer, and uterine cancer.

5. The method of claim 4 wherein the cancer is breast cancer.

6. The method of claim 1 wherein when it is determined that the subject suffers from cancer, a nucleic acid encoding for hGSTA1, a chemotherapeutic agent, a radiotherapeutic agent or an immunotherapeutic agent is administered to the subject.

7. The method of claim 3 wherein when it is determined that the subject will achieve a response with dendrogenin A, the subject is then administered with said dendrogenin A.

8. The method of claim 2, wherein the cancer is selected from the group consisting of bile duct cancer, bladder cancer, bone cancer, brain and central nervous system cancer, breast cancer, Castleman disease, cervical cancer, colorectal cancer, endometrial cancer, esophagus cancer, gallbladder cancer, gastrointestinal carcinoid tumors, Hodgkin's disease, non-Hodgkin's lymphoma, Kaposi's sarcoma, kidney cancer, laryngeal and hypopharyngeal cancer, liver cancer, lung cancer, mesothelioma, plasmacytoma, nasal cavity and paranasal sinus cancer, nasopharyngeal cancer, neuroblastoma, oral cavity and oropharyngeal cancer, ovarian cancer, pancreatic cancer, penile cancer, pituitary cancer, prostate cancer, retinoblastoma, rhabdomyosarcoma, salivary gland cancer, skin cancer, stomach cancer, testicular cancer, thymus cancer, thyroid cancer, vaginal cancer, vulvar cancer, and uterine cancer.

9. The method of claim 3, wherein the cancer is selected from the group consisting of bile duct cancer, bladder cancer, bone cancer, brain and central nervous system cancer, breast cancer, Castleman disease, cervical cancer, colorectal cancer, endometrial cancer, esophagus cancer, gallbladder cancer, gastrointestinal carcinoid tumors, Hodgkin's disease, non-Hodgkin's lymphoma, Kaposi's sarcoma, kidney cancer, laryngeal and hypopharyngeal cancer, liver cancer, lung cancer, mesothelioma, plasmacytoma, nasal cavity and paranasal sinus cancer, nasopharyngeal cancer, neuroblastoma, oral cavity and oropharyngeal cancer, ovarian cancer, pancreatic cancer, penile cancer, pituitary cancer, prostate cancer, retinoblastoma, rhabdomyosarcoma, salivary gland cancer, skin cancer, stomach cancer, testicular cancer, thymus cancer, thyroid cancer, vaginal cancer, vulvar cancer, and uterine cancer.
